SEQUENCE LISTING (30 sequences)

SEQ ID NO: 1 - DNA, 245 bp, Histophilus somni strain 8025
tacggagggtgcgagcgttaatcggaatgactgggcgtaaagggcacgcaggtggtgacttaagtgaggtgtgaaatccccgggcttaacctgggaacagcatttcagactgggtgactagagtactttagggaggggtagaattccacgtgtagcggtgaaatgcgtagagatgtggaggaataccgaaggcgaaggcagccccttgggaagatactgacgctcgagtgcgaaagcgtggggag

SEQ ID NO: 2 - DNA, 245 bp, Colidextribacter massiliensis strain Marseille-P3083
tacgtaggtggcaagcgttatccggatttactgggtgtaaagggcgcgtaggcggggatgcaagtcagatgtgaaatctatgggctcaactcataaactgcatttgaaactgtatctcttgagtgctggagaggtagatggaattccttgtgtagcggtgaaatgcgtagatataaggaagaacaccagtggcgaaggcgatctactggacagtaactgacgctgaggcgcgagagcgtggggag

SEQ ID NO: 3 - DNA, 245 bp, Campylobacter lanienae strain CCUG 44467
tacggagggtgcaagcgttactcggaatcactgggcgtaaaggacgcgtaggcggattatcaagtcttttgtgaaatctaatggctcaaccattaaactgcttgagaaactgataatctagagtgagggagaggcagatggaattggtggtgtaggggtaaaatccgtagagatcaccaggaatacccattgcgaaggcgatctgctggaacttaactgacgctaatgcgtgaaagcgtggggag

SEQ ID NO: 4 - DNA, 245 bp, Oscillibacter ruminantium strain GH1
tacgtaggtggcaagcgttgtccggatttactgggtgtaaagggcgtgtagccgggtcggtaagtcagatgtgaaatctggaggctcaacctccaaactgcatttgaaactgctgatcttgagtatcggagaggcaatcggaattcctagtgtagcggtgaaatgcgtagatattaggaggaacaccagtggcgaaggcggattgctggacgacaactgacggtgaggcgcgaaagcgtggggag

SEQ ID NO: 5 - DNA, 245 bp, Bacteroides plebeius strain M12
tacggaggatgcgagcgttatccggatttattgggtttaaagggagcgtagacgggagattaagtcagctgtgaaagtttgaggcccaaccttaaaattgcagttgatactggtttccttgagtgcggttgaggtgtgtggaattcgtggtgtagcggtgaaatgcttagatatcacgaagaactccgattgcgaaggcagcacactaagccgtaactgacgttgaggctcgaaagtgtgggtat

SEQ ID NO: 6 - DNA, 245 bp, Bacteroides plebeius strain M12
tacggaggatgcgagcgttatccggatttattgggtttaaagggagcgtagacgggagattaagtcagttgtgaaaggctgcggctcaaccgcagcactgcagttgatactggtttccttgagtgcggttgaggtgtatggaattcgtggtgtagcggtgaaatgcttagatatcacgaagaactccgattgcgaaggcagtacactaagccgtaactgacgttgaggctcgaaagtgtgggtat

SEQ ID NO: 7 - DNA, 245 bp, Ihubacter massiliensis strain Marseille-P2843
tacgtagggggcaagcgttatccggaatcattgggcgtaaagagtacgtaggtggttacctaagcaaggggtttaaggcaatggctcaaccattgttcgccccttgaactgggctacttgagtgcaggagaggaaagcggaattcctagtgtagcggtgaaatgcgtagatattaggaggaacaccagtggcgaaggcggctttctggactgtaactgacactgaggtacgaaagcgtggggagc

SEQ ID NO: 8 - DNA, 245 bp, Colidextribacter massiliensis strain Marseille-P3083
tacgtaggtggcgagcgttatccggatttactgggtgtaaagggtgtgtaggcgggctggtaagtcagatgtgaaaactatgggcctaacccatagcctgcatttgaaactatcggtcttgagagtgggagaggtaaatggaattcccggtgtagcggtgaaatgcgtagatatcgggaggaacaccagtggcgaaggcgatttactggaccacaactgacgctgagacacgaaagcgtggggag

SEQ ID NO: 9 - DNA, 245 bp, Bacteroides plebeius strain M12
tacggaggatgcgagcgttatccggatttattgggtttaaagggagcgtagacgggagattaagtcagctgtgaaagtttgaggcccaaccttaaaattgcagttgatactggtttccttgagtgcggttgaggtgtatggaattcgtggtgtagcggtgaaatgcttagatatcacgaagaactccgattgcgaaggcagtacactaagccgtaactgacgttgaggctcgaaagtgtgggtat

SEQ ID NO: 10 - DNA, 245 bp, Intestinimonas butyriciproducens strain SRB-521-5-I
tacgtaggtggcgagcgttatccggatttactgggtgtaaagggcgcgtaggcgggggtgcaagtcagatgtgaaatctatgggctcaactcataaactgcatttgaaactgtacttcttgagtactggagaggtagacggaattccttgtgtagcggtgaaatgcgtagatataaggaagaacaccagtggcgaaggcggtctactggacagcaactgacgctgaggcgcgaaagcgtggggag

SEQ ID NO: 11 - DNA, 245 bp, Bacteroides xylanolyticus strain X5-1
tacgtagggggcaagcgttatccggatttactgggtgtaaagggagcgtagacggttgtgcaagtctgatgtgaaagcccggtgctcaacgccgggactgcattggaaactgtataactagagtgcaggagaggtaagcggaattcctagtgtagcggtgaaatgcgtagatattaggaggaacaccagtggcgaaggcggcttactggactgtaactgacgttgaggctcgaaagcgtggggag

SEQ ID NO: 12 - DNA, 245 bp, Merdimonas faecis strain BR31
tacgtagggggcaagcgttatccggatttactgggtgtaaagggagcgtagacggcgaggcaagtccgatgtgaaaacccggggctcaaccccgggattgcattggaaactgtttggctcgagtactggaggggcaagcggaattcctggtgtagcggtgaaatgcgtagatatcaggaggaacaccggtggcgaaggcggcttgctggacagtgactgacgttgaggctcgaaagcgtggggag

SEQ ID NO: 13 - DNA, 245 bp, Ruminococcus lactaris strain ATCC 29176
tacgtagggggcaagcgttatccggatttactgggtgtaaagggagcgcagacggaactgcaagtctgatgtgaaagcccggggctcaaccgcgggactgcatcggatactgtagatctggagtgccggaggggcaagcggaattcctggtgtagcggtgaaatgcgtagatatcaggaggaacaccggcggcgaaggcggcttgctggacggcaactgacgttgaggctcgaaagcgtggggag

SEQ ID NO: 14 - DNA, 245 bp, [Clostridium] cellobioparum strain DSM 1351
tacgtaggttgcaagcgttgtccggaattactgggtgtaaagggcgtgtaggcggagatgcaagttgggagtgaaatccatgggctcaacccatgaactgctttcaaaactgcatcccttgagtatcggagaggcaagcggaattcctagtgtagcggtgaaatgcgtagatattaggaggaacaccagtggcgaaggcggcttgctggacgacaactgacgctgaggcgcgaaagcgtggggag

SEQ ID NO: 15 - DNA, 245 bp, Lactonifactor longoviformis strain ED-Mt61/PYG-s6
tacgtagggggcaagcgttatccggatttactgggtgtaaagggagcgcagacggcagtgcaagtctgatgtgaaagcccggggctcaaccccggaactgcattggaaactgtacagcttgagtgtcggagaggtaagcggaattcctagtgtagcggtgaaatgcgtagatattaggaggaacaccggtggcgaaggcggcttactggacgatgactgacgttgaggctcgaaagcgtggggag

SEQ ID NO: 16 - DNA, 245 bp, Parapedobacter lycopersici strain T16R-256
tacgggggatgcgagcgttatccggatttattgggtttaaagggtgcgtaggcggcctgttaagtcagcggtgaaattcaggggctcaacctctgccgtgccgttgatactggcgagctagaatgcggatgccgttggaggaatgtgtggtgtagcggtgaaatgcatagatatcacacagaacaccgattgcgaaggcatctgacgaatccgctattgacgctgatgcacgaaagcgtggggat

SEQ ID NO: 17 - DNA, 245 bp, Parapedobacter soli strain DCY14
tacgggggatgcaagcgttatccggatttattgggtttaaagggtgcgtaggcggtcaggtaagtcagcggtgaaatttaggggctcaacctctaccgtgccgttgatactgtctgactagaatgcggatgccgtgggaggaatgtgtggtgtagcggtgaaatgcatagatatcacacagaacaccgattgcgaaggcatctcacgaatccgcaattgacgctgaggcacgaaagcgtggggat

SEQ ID NO: 18 - DNA, 245 bp, [Clostridium] hylemonae strain TN-271
tacgtagggggcaagcgttatccggatttactgggtgtaaagggagcgtagacggtcttgcaagtctgaagtgaaagcccggggctcaaccccgggactgctttggaaactgtatgactagagtgtcggagaggcaagtggaattcctagtgtagcggtgaaatgcgtagatattaggaggaacaccagtggcgaaggcggcttgctggacgatgactgacgttgaggctcgaaagcgtggggag

SEQ ID NO: 19 - DNA, 245 bp, Bacteroides mediterraneensis strain Marseille-P2644
tacggaggatgcgagcgttatccggatttattgggtttaaagggagcgtagacgggatgttaagtcagttgtgaaaggctgcggctcaaccgcagcactgcagttgatactggcgtccttgagtgcggttgaggtatgtggaattcgtggtgtagcggtgaaatgcttagatatcacgaagaactccgattgcgaaggcagcatactaagccgctactgacgttgaggctcgaaagtgtgggtat

SEQ ID NO: 20 - DNA, 245 bp, Enterorhabdus muris strain WCA-131-CoC-2
cacgtagggggcgagcgttatccggattcattgggcgtaaagcgcgcgcaggcggccgctcaagcgggacctctaatcccggggctcaaccccgggccgggtcccgaactgggcggctcgcgtcaggtagggggaggcggaattcccggtgtagcggtggaatgcgcagatatcgggaggaacaccgacggcgaaggcagcctcctgggcctcgacggacgctgaggcgcgaaagccgggggagc

SEQ ID NO: 21 - DNA, 245 bp, Eubacterium pyruvativorans strain I-6
tacgtagggggcgagcgttatccggaattactgggcgtaaagagtgcgtaggtggtcctgcaagcgcggaggtctaaggctacggcctaaccgtagttcgccccgcgaactgcagaacttgagtacaggaggggaaagcggaattcctagtgtagcggtgaaatgcgtagatattaggaggaacaccagtggcgaaggcggctttctggactgcaactgacactgaggcacgaaagcgtgggtag

SEQ ID NO: 22 - DNA, 245 bp, Monoglobus pectinilyticus strain 14
tacgtaggtggcaagcgttgtccggatttactgggtgtaaagggcgcgtaggcgggtctttaagtcaggtgtgaaatccctcggctcaaccgaggaactgcacttgaaactggagatcttgagtgtcggagagggaagcggaattcctagtgtagcggtgaaatgcgtagatattaggaggaacaccagtggcgaaggcggctttctggacgataactgacgctgaggcgcgaaagcgtggggag

SEQ ID NO: 23 - DNA, 245 bp, Cloacibacillus porcorum strain CL-84
tacgtagggggcgagcgttgttcggaattactgggcgtaaagcgcacgcaggctggccgttaagtctgtcgtcaaaggcgggggctcaacctccgttctacgatagatactggcggcctagagtatgtgagagggaagtggaattcccggtgtagcggtgaaatgcgtagatatcgggaggaacaccagtggcgaaggcggcttcctggcacacaactgacgctcatgtgcgaaagccagggcag

SEQ ID NO: 24 - DNA, 245 bp, Monoglobus pectinilyticus strain 14
tacgtaggttgcaagcgttgtccggatttactgggtgtaaagggcgtgtaggcggagatgcaagttgggagtgaaatcccggggctcaaccccggaactgctttcaaaactgtatcccttgagtgtcggagaggcaagcggaattcctagtgtagcggtgaaatgcgtagatattaggaggaacaccagtggcgaaggcggcttgctggacgataactgacgctgaggcgcgaaagcgtggggag

SEQ ID NO: 25 - DNA, 245 bp, Harryflintia acetispora strain V20-281a
tacgtaggtggcaagcgttgtccggaattactgggtgtaaagggagcgcaggcgggactgcaagttggatgtgaaataccgcagcttaactgcggagctgcatccaaaactgtagttcttgagtggagtagaggtaggcggaattccgagtgtagcggtgaaatgcgtagatattcggaggaacaccagtggcgaaggcggcctactgggctcttactgacgctgaggctcgaaagtgtggggag

SEQ ID NO: 26 - DNA, 245 bp, Collinsella massiliensis strain GD3
tacgtatggggcgagcgttatccggaatcattgggcgtaaagcgcgcgtaggcggctgtccaagcggggcgtcaaatcccggggctcaaccccgggccgcgccccgaactgggcggcttgagtccggtagggggaggcggaattcccggtgtagcggtgaaatgcgcagatatcgggaagaacaccggtggcgaaggcggcctcccgggccgcgactgacgctgaggcgcgaaagctaggggagc

SEQ ID NO: 27 - DNA, 245 bp, Monoglobus pectinilyticus strain 14
tacgtaggtggcaagcgttgtccggatttactgggtgtaaagggcgtgtaggcgggtgagtaagtcagatgtgaaataccgaggctcaacctcggggctgcatttgaaactacttatcttgagtatcggagaggaaagcggaattcccagtgtagcggtgaaatgcgtagatattgggaggaacaccagtggcgaaggcggctttctggacgacaactgacgctgaggcgcgaaagcgtgggtag

SEQ ID NO: 28 - DNA, 245 bp, Denitrobacterium detoxificans strain NPOH1
tacgtagggagcgagcgttatccggattcattgggcgtaaagcgcgcgtaggcggctggccaagcgggatctcaaatccgggggctcaacctccggccggatcccgaactggccggctcgagttcggtagaggaagacggaattcccagtgtagcggtgaaatgcgcagatattgggaagaacaccgatggcgaaggcagtcttctgggccgcgactgacgctgaggtgcgaaagccgggggagc

SEQ ID NO: 29 - DNA, 245 bp, Novibacillus thermophilus strain SG-1
tacgtagggggcaagcgttgtccggaatgattgggcgtaaagggcgcgtaggcggcctgttaagtctgaagtgaaactcccgcttttaaggtgggaactgctttggatactgatgggcttgagtgcaggagaggtaagcggaattcccggtgtagcggtgaaatgcgtagagatcgggaggaacaccagtggcgaaggcggcttactggactgtaactgacgctgaggcgcgaaagtgtggggag

SEQ ID NO: 30 - DNA, 245 bp, Monoglobus pectinilyticus strain 14
tacgtaggttgcaagcgttgtccggatttactgggtgtaaagggcgtgtaggcggagatgcaagttgggagtgaaatcccggggctcaaccccggaactgctttcaaaactgcatcccttgagtatcggagaggcaagcggaattcctagtgtagcggtgaaatgcgtagatattaggaggaacaccagtggcgaaggcggcttgctggacgacaactgacgctgaggcgcgaaagcgtggggag

(19) United States
(12) Patent Application Publication
(10) Pub. No.: US 2021/0032687 A1
(43) Pub. Date: Feb. 4, 2021

(54) VAGINAL AND FECAL MICROBIOME BIOMARKERS FOR PREDICTING BOVINE REPRODUCTIVE TRAITS

(71) Applicant: THE BOARD OF TRUSTEES OF THE UNIVERSITY OF ARKANSAS, Little Rock, AR (US)

(72) Inventors: Jiangchao Zhao, Fayetteville, AR (US); Feilong Deng, Fayetteville, AR (US); Rick Rorie, Springdale, AR (US); Maryanna M. Hudson, Starkville, MS (US)

(73) Assignee: THE BOARD OF TRUSTEES OF THE UNIVERSITY OF ARKANSAS, Little Rock, AR (US)

(21) Appl. No.: 16/943,690

(22) Filed: Jul. 30, 2020

Related U.S. Application Data
(60) Provisional application No. 62/880,472, filed on Jul. 30, 2019.

(51) Int. Cl. C12Q 1/689; C12Q 1/6869; G16B 30/00

(52) U.S. Cl. CPC C12Q 1/689; G16B 30/00; C12Q 1/6869

(57) ABSTRACT

To gain insights into the relationship between microbiota and fertility, the vaginal and fecal microbiomes of female cows were examined throughout pregnancy. Next generation sequencing and Random Forest modeling were used to identify bacterial biomarkers present in vaginal and fecal samples that are predictive of pregnancy status. The present invention provides methods and kits that can be used to select female cows to include in a breeding program based on detection of these biomarkers.

### CROSS-REFERENCE TO RELATED APPLICATIONS

This patent application claims the benefit of priority of United States Provisional Patent Application No. 62/880,472, filed Jul. 30, 2019, which is incorporated herein by reference in its entirety.

### SEQUENCE LISTING

A Sequence Listing accompanies this application and is submitted as an ASCII text file of the sequence listing named “169946_00566_ST25.txt” which is 14.6 KB in size and was created on Jul. 30, 2020. The sequence listing is electronically submitted via EFS-Web with the application and is incorporated herein by reference in its entirety.

### INTRODUCTION

Reproduction has an enormous impact on profitability in commercial cattle operations¹. The combined income losses due to reproductive failure in the dairy and beef industries total $1 billion annually, making reproductive failure six times more costly than losses associated with respiratory disease². Although a myriad of other factors contribute to this financial loss, national losses due to culling infertile females alone average $249 million annually². Attention to nutrition and seasonality and the use of reproductive technologies and management strategies involving genetic selection can improve reproductive efficiency in a beef herd³⁻⁵. Still, methods to predict the ability of heifers to establish pregnancy would dramatically reduce costs related to reproductive failure.

The interrelationship between hosts and their microbes is important in female fertility. In humans and non-human species alike, either suppression or overgrowth of certain bacterial species in a particular niche can result in disease, emphasizing the importance of understanding the ways the host environment and inhabiting microbes interact¹⁷⁻²⁰. It was previously reported that Lactobacillus dominance is crucial to vaginal health in humans, but not in other species¹⁴. Studies in which probiotics were used to shift microbial communities in gestating humans have shown positive outcomes. Here, communities were manipulated to inhibit the growth of microbes that modify the host inflammatory response and signal for pre-term birth²¹. When ingested, these live organisms can stimulate the vaginal and gut microbiomes to produce metabolites and other products that promote favorable metabolic activity during the late stages of gestation²². Understanding the role that certain species of bacteria play in fertility and reproductive performance in female cattle could help increase reproductive fitness in herds worldwide.

Several studies have profiled the microbial composition of the bovine vagina. A study by Swartz et al.¹⁵ reported that this niche is dominated by Aggregatibacter, Streptobacillus, Phocoenobacter, Sediminicola and Sporobacter species, while a study reported by Gonzalez and colleagues¹⁶ listed members of Firmicutes, Bacteroidetes, Ruminococcus, Dialister, Aeribacillus, and Porphyromonas as the dominant colonizers. Importantly, differences in the relative abundance of certain genera within the vaginal microbiome have been linked to reproductive disorder in female bovine. Increased relative abundances of members of Bacteroides and Enterobacteriaceae (35.83% and 18.62%, respectively) have been shown in females with reproductive disease as compared to healthy females (28.3% and 17.8%, respectively)¹⁷. Histophilus has also been isolated from vaginal communities in cattle with reproductive disorders, and not from those of healthy cattle¹⁷.

Thus, there is a pressing need in the cattle industry for methods to reduce costs related to reproductive failure, and better understanding of the relationship between vaginal microbiota and fertility is likely to aid in this effort.

### SUMMARY

In the present application, the vaginal microbiome of commercial beef heifers was characterized over the course of pregnancy. Because the gut microbiome is known to play an important role in health and disease²³, the fecal microbiome of pregnant heifers was also characterized. The inventors demonstrate herein that the presence, absence, or level of particular bacteria in the vagina or fecal matter from a female cow is indicative of the breeding success of that cow.

The present invention provides methods for selecting female cows to include in a breeding program. The female cows are selected for a relatively high rate of breeding success on the first attempt (i.e., carrying a calf to full gestation with a live birth). The methods include collecting a vaginal swab or a fecal sample from a female cow, measuring the level of at least one biomarker associated with at least one bacterium, and analyzing the abundance of the biomarker to determine whether to breed the female cow.

For the vaginal swab samples, the level of a biomarker associated with a bacterium of a species selected from Histophilus somni, Colidextribacter massiliensis, Campylobacter lanienae, Bacteroides plebeius, Bacteroides xylanolyticus, Ihubacter massiliensis, Intestinimonas butyriciproducens, Merdimonas faecis, Ruminococcus lactaris, Lactonifactor longoviformis, Oscillibacter ruminantium, and [Clostridium] cellobioparum is indicative of the likelihood of successful breeding and the abundance of the biomarker in the sample relative to control cows is analyzed to determine whether to breed the female cow. The female cow is bred if one or more of the following differences in the abundance of a biomarker associated with a bacterial species is detected as compared to control cows: a decrease in Histophilus somni, decrease in Colidextribacter massiliensis, decrease in Campylobacter lanienae, decrease in Bacteroides xylanolyticus, decrease in Ihubacter massiliensis, decrease in Intestinimonas butyriciproducens, decrease in Merdimonas faecis, decrease in Ruminococcus lactaris, decrease in Lactonifactor longoviformis, increase in Oscillibacter ruminantium, or increase in [Clostridium] cellobioparum.

In some aspects of the invention, the biomarker measured in the vaginal swab sample is associated with a bacterium of one or more of following strains: Histophilus somni strain 8025, Colidextribacter massiliensis strain Marseille-P3083, Campylobacter lanienae strain CCUG, Oscillibacter ruminantium strain GH1, Bacteroides plebeius strain M12, Ihubacter massiliensis strain Marseille, Intestinimonas butyriciproducens strain SRB-521-5-I, Bacteroides xylanolyticus strain X5-1, Merdimonas faecis strain BR31, Ruminococcus lactaris strain ATCC, [Clostridium] cellobioparum strain DSM 1351, and Lactonifactor longoviformis strain ED-Mt61/PYG-s6.

For the fecal samples obtained from a female cow, the level of a biomarker associated with a bacterium of a species selected from Bacteroides mediterraneensis, Enterorhabdus muris, Eubacterium pyruvativorans, Monoglobus pectinilyticus, Harryflintia acetispora, Collinsella massiliensis, Denitrobacterium detoxificans, Parapedobacter lycopersici, Parapedobacter soli, [Clostridium] hylemonae, Cloacibacillus porcorum, and Novibacillus thermophiles is indicative of the likelihood of successful breeding and the abundance of the biomarker as compared to control cows is analyzed to determine whether to breed the female cow. The female cow is bred if one or more of the following differences in the abundance of a biomarker associated with a bacterial species is detected as compared to control cows: a decrease in Bacteroides mediterraneensis, decrease in Enterorhabdus muris, decrease in Eubacterium pyruvativorans, decrease in Harryflintia acetispora, decrease in Collinsella massiliensis, decrease in Denitrobacterium detoxificans, increase in Parapedobacter lycopersici, increase in Parapedobacter soli, increase in [Clostridium] hylemonae, increase in Cloacibacillus porcorum, or an increase in Novibacillus thermophiles.

In some aspects of the invention, the biomarker measured in the fecal sample is associated with a bacterium of one or more of following strains: Parapedobacter lycopersici strain T16R-256, Parapedobacter soli strain DCY14, [Clostridium] hylemonae strain TN-271, Bacteroides mediterraneensis strain Marseille-P2644, Enterorhabdus muris strain WCA-131-CoC-2, Eubacterium pyruvativorans strain 1-6, Monoglobus pectinilyticus strain 14, Cloacibacillus porcorum strain CL-84, Harryflintia acetispora strain V20-281a, Collinsella massiliensis strain GD3, Denitrobacterium detoxificans strain NPOH1, and Novibacillus thermophiles strain SG-1.

In some aspects of the invention, the step of measuring the level of a biomarker comprises detecting a protein associated with a particular bacterium using for example an antibody-based method.

In other aspects of the invention, the step of measuring the level of a biomarker comprises detecting a nucleic acid, such as RNA or DNA, associated with a particular bacterium. In some aspects, the nucleic acid is a component of a 16S or 23S ribosomal subunit, and in certain cases, the nucleic acid comprises a V4 region of a 16S rRNA gene selected from the group consisting of SEQ ID NOs: 1-30. The nucleic acids may be measured or detected by extracting nucleic acid from a sample and using at least one set of PCR primers to amplify and detect the nucleic acids.

In some aspects of the invention, the sample is collected from a female cow at the onset of the breeding season, prior to breeding, or prior to estrus synchronization.

The present invention further provides kits comprising reagents used to detect the presence or relative abundance of at least 2 biomarkers associated with bacteria of the following species in vaginal swab samples collected from a female cow: Histophilus somni, Colidextribacter massiliensis, Campylobacter lanienae, Bacteroides plebeius, Bacteroides xylanolyticus, Ihubacter massiliensis, Intestinimonas butyriciproducens, Merdimonas faecis, Ruminococcus lactaris, Lactonifactor longoviformis, Oscillibacter ruminantium, and [Clostridium] cellobioparum.

In some aspects of the invention, at least one of the measured biomarkers is associated with a bacterium of the following strains: Histophilus somni strain 8025, Colidextribacter massiliensis strain Marseille-P3083, Campylobacter lanienae strain CCUG, Oscillibacter ruminantium strain GH1, Bacteroides plebeius strain M12, Ihubacter massiliensis strain Marseille, Intestinimonas butyriciproducens strain SRB-521-5-I, Bacteroides xylanolyticus strain X5-1, Merdimonas faecis strain BR31, Ruminococcus lactaris strain ATCC, [Clostridium] cellobioparum strain DSM 1351, or Lactonifactor longoviformis strain ED-Mt61/PYG-s6.

In certain aspects of the invention, the presence or absence of the bacterial species Campylobacter lanienae, Merdimonas faecis, or Lactonifactor longoviformis is assessed qualitatively. Female cows having any of these bacteria in their vaginal swabs should not be bred, as they are unlikely to breed successfully.

The present invention also provides kits comprising reagents used to detect the presence or relative abundance of at least 2 biomarkers associated with bacteria of the following species in fecal samples collected from a female cow: Bacteroides mediterraneensis, Enterorhabdus muris, Eubacterium pyruvativorans, Monoglobus pectinilyticus), Harryflintia acetispora, Collinsella massiliensis, Denitrobacterium detoxificans, Parapedobacter lycopersici, Parapedobacter soli, [Clostridium] hylemonae, Cloacibacillus porcorum, and Novibacillus thermophiles.

In some aspects of the invention, at least one of the measured biomarkers is associated with a bacterium of the following strains: Parapedobacter lycopersici strain T16R-256, Parapedobacter soli strain DCY14, [Clostridium] hylemonae strain TN-271, Bacteroides mediterraneensis strain Marseille-P2644, Enterorhabdus muris strain WCA-131-CoC-2, Eubacterium pyruvativorans strain 1-6, Monoglobus pectinilyticus strain 14, Cloacibacillus porcorum strain CL-84, Harryflintia acetispora strain V20-281a, Collinsella massiliensis strain GD3, Denitrobacterium detoxificans strain NPOH1, or Novibacillus thermophiles strain SG-1.

In certain aspects of the invention, the presence or absence of the bacterial species Eubacterium pyruvativorans, Monoglobus pectinilyticus or Cloacibacillus porcorum is assessed qualitatively. Female cows having Eubacterium in their fecal sample should not be bred, as they are unlikely to breed successfully. Female cows having Monoglobus or Cloacibacillus should be bred, as they are likely to breed successfully.

In some aspects of the invention, the kit further comprises antibodies or PCR primers specific to proteins associated with particular bacteria. The nucleic acids may be components of a 16S or 23S ribosomal subunit, and in certain cases the nucleic acids comprise at least one sequence selected from the group consisting of SEQ ID NOs: 1-30.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIG. 1 shows boxplots comparing the alpha diversity measured in the vaginal microbial community between bred and open female cattle at four stages of pregnancy (indicated below) based on Shannon index (A) and observed operational taxonomic units (OTUs) (B). The bottom and top of each box are the first and third quartiles, respectively, and the band inside the box is the median. Bred: cattle that were pregnant after the breeding season; Open: cattle that never established pregnancy.

FIG. 2 shows boxplots comparing the alpha diversity measured in the fecal microbial community between bred and open female cattle at the indicated stage of pregnancy (indicated below) based on Shannon index (A) and observed OTUs (B). The bottom and top of each box are the first and third quartiles, respectively, and the band inside the box is the median. Bred: cattle that were pregnant after the breeding season; Open: cattle that never established pregnancy.

FIG. 3 shows Principal Coordinate Analysis (PCoA) plots comparing the beta diversity measured in vaginal (A and B) and fecal (C and D) samples across gestation stages between open and bred cattle. Panels A and C show the PCoA plot based on community membership as measured by Jaccard distance. Panels B and D show the PCoA plot based on community structure as measured by Bray-Curtis dissimilarity matrices. Triangles and circles represent bred and open females, respectively. Stages are indicated by color: red, blue, green, and purple represent pre-breeding, and gestational trimesters 1 through 3, respectively. The ellipses represent 0.95 confidence intervals. Bred: cattle that were pregnant after the breeding season; Open: cattle that never established pregnancy.

FIG. 4 shows multi-colored stacked bar graphs representing the relative abundance of the top 15 bacterial features in the vaginal microbiome of beef heifers that are predictive of pregnancy status. Panels A-D show the relative abundance of bacteria at the species level. Each panel shows a different stage of pregnancy (A: pre-breeding, B: first trimester, C: second trimester, D: third trimester) and each bar represents a sample. Panels E-H show the relative abundance of bacteria at the phylum level. Each panel shows a different stage of pregnancy (E: pre-breeding, F: first trimester, G: second trimester, H: third trimester) and each bar represents a sample.

FIG. 5 shows multi-colored stacked bar graphs representing the relative abundance of the top 15 bacterial features in the fecal microbiome of beef heifers that are predictive of pregnancy status. Panels A-B show the relative abundance of bacteria at the species level. Each panel shows a different stage of pregnancy (A: pre-breeding, B: first trimester) and each bar represents a sample. Panels C-D show the relative abundance of bacteria at the phylum level. Each panel shows a different stage of pregnancy (C: pre-breeding, D: first trimester) and each bar represents a sample.

FIG. 6 shows the top vaginal bacterial features that are predictive of pregnancy status. Panel A shows the ROC curve of the optimal random forest model created by comparing the vaginal microbiota of bred and open beef heifers at the pre-breeding stage. Panel B shows the top 15 features identified by the random forest model. Panels C-E compare the relative abundance of the top three most predictive features in bred and open heifers.

FIG. 7 shows the top fecal bacterial features that are predictive of pregnancy status. Panel A shows the ROC curve of the optimal random forest model created by comparing the fecal microbiota of bred and open beef heifers at the pre-breeding stage. Panel B shows the top 15 features identified by the random forest model Panels C-E compare the relative abundance of the top three most predictive features in bred and open heifers.

### DETAILED DESCRIPTION

In recent years, tremendous efforts have been made to explore microbiomes from all over human and animal bodies. Studies in which metagenomic data is accompanied by a host phenotype have allowed associations to be made between microbial profiles and traits of interest. A number of characteristics, such as species abundance and community diversity, have now been associated with particular diseases or health statuses.

The interrelationship between hosts and their microbes is known to be important in female fertility. In cows, differences in the abundance of certain bacteria in the vaginal microbiome have been linked to reproductive disorders. As the inventors demonstrate in the Examples, the bacterial profile of both the vaginal and fecal microbiome can be used to predict whether a heifer will establish pregnancy upon breeding. Here, sets of bacterial features that can serve as biomarkers for the ability to become pregnant were identified from each of these microbial niches. While the presence or increased level of some of these biomarkers indicates that a subject is likely to become pregnant, the absence or decreased level of other biomarkers indicates that a subject is likely to become pregnant. The opposite is also true: the presence or increased abundance of certain biomarkers may indicate that the subject is unlikely to become pregnant, while the absence or decreased abundance of other biomarkers is indicative that the subject is unlikely to become pregnant.

The present invention provides methods and kits for assigning female cows to be bred or culled based on the levels of these biomarkers in the vaginal or fecal microbiome of the cows. In some embodiments, the assessment of certain biomarkers will be qualitative (i.e. based simply on whether it is present in the sample at detectable levels or not), while the assessment of other biomarkers will be relative to the levels in a control sample. A control sample as used herein is based on a mean level of these markers in the vaginal and fecal samples from female cows. In some embodiments, a sample of the vaginal microbiome is obtained by vaginal swab.

In other embodiments, a sample of the gut microbiome is obtained by collecting a fecal sample. Those skilled in the art are familiar with the methods for collection, maintenance, and preparation of such samples. The vaginal swab or fecal sample may be taken prior to the cow entering estrus or during estrus. The vaginal swab or fecal sample may also be taken before or during estrus synchronization. The vaginal or fecal sample may be obtained prior to breeding.

In embodiments utilizing a vaginal swab sample, the measured biomarkers are associated with bacteria of a species from the group consisting of: Histophilus somni, Colidextribacter massiliensis, Campylobacter lanienae, Bacteroides plebeius, Bacteroides xylanolyticus, Ihubacter massiliensis, Intestinimonas butyriciproducens, Merdimonas faecis, Ruminococcus lactaris, Lactonifactor longoviformis, Oscillibacter ruminantium, and [Clostridium] cellobioparum. Here, the heifer will be bred if one or more of the following differences in the abundance of a bacterial species is detected: a decrease in Histophilus somni, decrease in Colidextribacter massiliensis, decrease in Campylobacter lanienae, decrease in Bacteroides xylanolyticus, decrease in Ihubacter massiliensis, decrease in Intestinimonas butyriciproducens, decrease in Merdimonas faecis, decrease in Ruminococcus lactaris, decrease in Lactonifactor longoviformis, increase in Oscillibacter ruminantium, or an increase in [Clostridium] cellobioparum. If the presence of a biomarker associated with at least one of Campylobacter, Merdimonas and Lactonifactor is not detected, then the cow should be bred. If the presence of a biomarker associated with at least one of Campylobacter, Merdimonas and Lactonifactor is detected, then the cow should not be bred. If any two or all three of Campylobacter, Merdimonas and Lactonifactor is not detected, then the cow should be bred and if any two or all three are detected, then the cow should not be bred.

In certain embodiments, the biomarkers measured in the vaginal microbiome are associated with bacteria that belong to one or more of the following strains: Histophilus somni strain 8025, Colidextribacter massiliensis strain Marseille-P3083, Campylobacter lanienae strain CCUG, Oscillibacter ruminantium strain GH1, Bacteroides plebeius strain M12, Ihubacter massiliensis strain Marseille, Intestinimonas butyriciproducens strain SRB-521-5-I, Bacteroides xylanolyticus strain X5-1, Merdimonas faecis strain BR31, Ruminococcus lactaris strain ATCC, [Clostridium] cellobioparum strain DSM 1351, or Lactonifactor longoviformis strain ED-Mt61/PYG-s6.

In other embodiments relying on fecal collection, the measured biomarkers are associated with bacteria of a species from the group consisting of: Bacteroides mediterraneensis, Enterorhabdus muris, Eubacterium pyruvativorans, Monoglobus pectinilyticus), Harryflintia acetispora, Collinsella massiliensis, Denitrobacterium detoxificans, Parapedobacter lycopersici, Parapedobacter soli, [Clostridium] hylemonae, Cloacibacillus porcorum, and Novibacillus thermophiles. Here, the heifer will be bred if one or more of the following differences in the abundance of a bacterial species is detected: a decrease in Bacteroides mediterraneensis, decrease in Enterorhabdus muris, decrease in Eubacterium pyruvativorans, decrease in Harryflintia acetispora, decrease in Collinsella massiliensis, decrease in Denitrobacterium detoxificans, increase in Parapedobacter lycopersici, increase in Parapedobacter soli, increase in [Clostridium] hylemonae, increase in Cloacibacillus porcorum, or an increase in Novibacillus thermophiles. If the biomarker associated with Eubacterium is not detected, then the female cow is bred. If the biomarker associated with the presence of at least one of Monoglobus and Cloacibacillus is detected, then the female cow is bred.

In certain embodiments, the biomarkers measured in the fecal microbiome are associated with bacteria that belong to one or more of the following strains: Parapedobacter lycopersici strain T16R-256, Parapedobacter soli strain DCY14, [Clostridium] hylemonae strain TN-271, Bacteroides mediterraneensis strain Marseille-P2644, Enterorhabdus muris strain WCA-131-CoC-2, Eubacterium pyruvativorans strain 1-6, Monoglobus pectinilyticus strain 14, Cloacibacillus porcorum strain CL-84, Harryflintia acetispora strain V20-281a, Collinsella massiliensis strain GD3, Denitrobacterium detoxificans strain NPOH1, or Novibacillus thermophilus strain SG-1.

The vaginal and fecal microbiome bacteria described above and in the Examples were classified based on current classifications of bacteria from the Ribosomal Database Project [17] using ribosomal RNA gene sequencing data. Those of skill in the art will appreciate that the names and strain designations of bacteria sometimes change over time as more information becomes available. Thus, the present application also provides the specific ribosomal sequences that were detected in the samples in addition to the names of the bacterial strains that were associated with these sequences at the time these experiments were completed. The ribosomal sequences identified in the vaginal microbiome are listed in Table 3 (SEQ ID NOs: 1-15) and the ribosomal sequences identified in the fecal microbiome are listed in Table 4 (SEQ ID NOs: 16-30).

The biomarkers utilized in the present invention may include any protein or nucleic acid that is specific to a relevant bacterium (described above) such that detection of the biomarker in a sample is indicative of the presence of that bacterium in the sample.

In some embodiments, the biomarkers are proteins that are associated with particular bacteria. Here, the biomarkers may be detected using antibodies that specifically recognize the bacterial proteins. Antibody-antigen recognition may be analyzed by a variety of methods known to those of skill in the art, including but not limited to ELISA (enzyme-linked immunosorbent assay), western blotting, dot blotting, immunohistochemistry, immunocytochemistry, fluorescence-activated cell sorting (FACS), immunoprecipitation, fluorescence microscopy, and protein microarray.

In other embodiments, the biomarkers are nucleic acids that are associated with particular bacteria. Nucleic acids can be extracted from a biological sample for analysis using standard techniques known in the art. In the present application, the terms “nucleic acid”, “polynucleotide”, and “oligonucleotide” are used interchangeably to refer to molecules of DNA and/or RNA. Methods for detecting nucleic acids may utilize one or more oligonucleotide probes or primers that selectively hybridize to a target nucleic acid that includes one or more of the biomarkers through complementary base pairing. As is known to those of skill in the art, a primer does not need to be perfectly complementary to a target sequence in order to hybridize with it, and it can be modified in a number of ways (e.g., methylation, fluorescent tagging) without altering the basic function of the primer.

In some embodiments, primers are used to detect the presence of nucleic acid biomarkers by amplification. Amplification-based methods include polymerase chain reaction (PCR) and primer extension reactions, wherein amplification of the product indicates the presence of the biomarker in the sample. The amplification product can be detected directly or indirectly by any method known in the art, including, but not limited to, visualization with ethidium bromide, label incorporation, and dye intercalation.

Other known hybridization-based methods of detection may also be utilized in the present invention. These methods generally rely on the detection of labeled probes (e.g., radioactively, fluorescently, and chemiluminescently labeled probes) that anneal to the target nucleic acid. Common hybridization-based methods include in situ hybridization, microarray analysis, oligonucleotide ligation assays, and Southern or northern blotting. In these methods, detection may involve comparing the amount of labeled probe that binds to target nucleic acid molecule as compared to a nucleic acid molecule other than the target molecule, particularly a substantially similar (i.e., homologous) nucleic acid molecule. Conditions that allow for selective hybridization can be determined empirically, or can be estimated based, for example, on the relative GC:AT content of the probe and the sequence to which it hybridizes, the length of the probe, or the number of mismatches between the probe and sequence to which it is to hybridize.

Many additional methods for detecting nucleic acids are known in the art and are encompassed by the present invention. These methods include those that rely on differential endonuclease digestion, such as restriction fragment length polymorphism (RFLP) analysis. Sequencing methods such as mass spectrometry, scanning electron microscopy, or methods in which a polynucleotide flows past a sorting device that can detect the sequence of the polynucleotide may also be utilized. For instance, in the Examples of the present invention, the biomarkers are detected using high-throughput sequencing followed by data analysis. Other formats may include electrochemical detection devices, melting curve analysis, and intercalating dyes. Useful methods include those that are readily adaptable to a high throughput format, to a multiplex format, or to both.

In certain embodiments of the invention, the biomarkers are measured quantitatively, to determine the abundance of the biomarkers in the microbiome sample relative to the abundance in a control sample. Quantitative methods of nucleic acid detection include, without limitation, arrays (e.g., microarrays), high-throughput sequencing, and real time PCR.

In some embodiments, the nucleic acid biomarkers are components of a ribosomal subunit. The sequences of ribosomal RNA (rRNA) genes, including 16S rRNA and 23S rRNA, are commonly used to identify and compare the bacteria or fungi present within a sample since they are found across nearly all forms of life. In certain embodiments, the nucleic acids comprise the V4 regions of 16S rRNA genes provided as SEQ ID NOs: 1-15 (Table 3) or SEQ ID NOs: 16-30 (Table 4) and utilized in the Examples.

The microbiome samples may be analyzed by individuals practicing the methods of the present invention, or alternatively, they may be analyzed by a separate entity, such as an independent testing laboratory.

Kits comprising reagents that may be used to detect the presence of the biomarkers of the present invention, described above, are also provided. In some embodiments, the kits are designed to detect the presence of biomarkers in vaginal swab samples. In other embodiments, the kits are designed to detect the presence of biomarkers in fecal samples. In certain embodiments, the presence of particular biomarkers is assessed qualitatively, while in other embodiments, the biomarkers are assessed quantitatively.

In some embodiments, the kits of the present invention comprise antibodies specific to proteins associated with particular bacteria. In other embodiments, the kits comprises sets of PCR primers that amplify nucleic acids associated with particular bacteria. In certain preferred embodiments, the kits use PCR primers to amplify nucleic acids that are components of the 16S or 23S ribosomal subunits of specific bacteria. However, the kits of the present invention may utilize any known method for detecting proteins or nucleic acids, including those detailed above.

The kits may contain additional reagents for performing methods described herein including, but not limited to, one or more detectable labels, which can be used to label a primer or can be incorporated into a product generated using primer (e.g., an amplification product); one or more polymerases, which can be useful for a method that includes a primer extension or amplification procedure; or other enzymes (e.g., a ligase or an endonuclease), which can be useful for performing an oligonucleotide ligation assay or a mismatch endonuclease cleavage assay; and/or one or more buffers or other reagents that are necessary to or can facilitate performing the methods. The primers can be included in a kit in a labeled form, for example with a label such as biotin or an antibody. The kits may also include instructions for performing the method or for analyzing the results and making predictions based on the results.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

### Identification of Biomarkers for the Ability to Establish Pregnancy in the Vaginal and Fecal Microbiomes

The interrelationship between hosts and their microbes is important in female fertility, and differences in the relative abundance of certain bacteria in the vaginal microbiome of female bovine have been linked to reproductive disorders. The bovine urogenital tract houses a variety of microbes composed of aerobic, facultative-anaerobic and anaerobic microorganisms³³. However, there is much variation in this niche due to intrinsic and extrinsic factors, and little is known about the roles microbes play in reproduction³⁴. In the present study, to gain insight into how microbes affect reproductive failure and success, the vaginal and fecal microbiome of commercial beef heifers were characterized and sets of biomarkers that predict whether or not a heifer will establish pregnancy were identified.

### Materials and Methods:

Ethics Statement:

All animal work was approved and all methods were performed in accordance with the guidelines of the Institutional Animal Care and Use Committee of the University of Arkansas under protocol #16024. The University of Arkansas Division of Agriculture's Beef Research Unit near Fayetteville, Ark., housed 72 crossbred beef heifers, averaging 420.88±17.42 days of age and 328.036±25.45 kg at the initiation of this study.

Breeding Strategy:

At the onset of the breeding season, a 25 mg PGF2α injection (Lutalyse®, Zoetis, Parsippany, N.J.) was administered intramuscularly into the neck of each heifer, and a heat detection patch (Estrotect Heat Patches®, Melrose, Minn.) was placed on the rump. Heifers were then allocated to one of six 1 hectare grass pastures. Each day for the next 7 days, all heifers were monitored for estrus activity at 8:30 am and 4:30 pm. Within 12 to 18 hours of estrus detection, heifers were artificially inseminated²⁴.

Seven days after estrus detection, individuals not showing signs of estrus like behavior were administered a second PGF2α injection. This group of heifers was monitored for five additional days and artificially inseminated, as described above. The heifers were then moved to six 2.4 hectare fescue-bermuda grass mixed pastures and were rotated every 28 days. Seven days after transfer to the pastures, a fertile bull was introduced to each pasture to initiate a 50-day breeding season. The bulls were rotated among the pastures every seven days. A breeding soundness examination was performed on each bull no more than 30 days before introduction to the heifer herd and following the 50-day breeding season to confirm fertility. After 50 days of exposure, all bulls passed breeding soundness examinations.

Sixty-three days after the onset of the breeding season, ultrasound was used to determine the heifer's pregnancy status and if the pregnancy was due to artificial insemination or natural breeding, based on a fetal crown to rump measurement.

Sample Collection:

At the onset of the breeding season, fecal samples were taken and immediately placed in 50 mL conical tubes on ice. The vulva was wiped clean with a paper towel and vaginal swabs were collected by inserting a double guarded culture swab (Jorgensen Labs, Loveland, Colo., USA) at a 45° angle into the vagina and moving it to the posterior cervix. At the posterior cervix, the swab and inner guard were maneuvered through the outer guard. The swab was then pushed out of the inner guard and rolled on the surface of the vaginal epithelium for approximately 15 seconds. The swab was then retracted back into the inner guard. The inner guard (containing the swab sample) was retracted into the outer guard and the double guarded swab was removed from the animal. The swab was cut from the handle, placed in a 2 mL snap-cap tube with 1 mL of AMIES transport buffer, and placed on ice. All samples were stored at −80° C. Fecal and vaginal samples were taken from all individuals, as described above, at a second time point during the first trimester of gestation. Vaginal swabs were also taken from all heifers during the second trimester of gestation and again for those with confirmed pregnancies during the third trimester of gestation.

Detailed health records were maintained for each heifer throughout the entirety of the trial to ensure that the health status of each individual was monitored. Each female was vaccinated and treated for external and internal parasites according to the University of Arkansas Division of Agriculture's Beef Research Unit cattle management protocol. Upon completion of the trial, pregnant heifers were retained as one group and open heifers were culled. The retained females were allowed to graze in fesuce-bermuda grass pastures and were supplemented with adequate free choice mineral supplements during gestation. Within 24 hours of birth, calf sex and birthweight were recorded²⁴.

DNA Extraction and Next-Generation Sequencing:

Approximately 0.1 g of thawed feces was used for DNA extraction with the QIAamp PowerFecal DNA Kit (QIAGEN Inc., Germantown, Md., USA) following the manufacturer's protocol. DNA was extracted from the vaginal swabs using the QIAAmp BiOStic Bacteremia DNA Kit (QIAGEN Inc., Germantown, Md., USA) according to the manufacturer's protocol. A Nanodrop One C (Fisher Scientific, Hanover Park, Ill., USA) was used to measure the DNA concentration and purity.

For library preparation, 10 ng of DNA were used for PCR amplification targeting the V4 region of the 16S rRNA gene. PCR was performed using dual index primers²⁵. Amplicons were normalized using a SequalPrep™ Normalization Kit (Life Technologies, Grand Island, N.Y., USA) according to the manufacturer's protocol. To generate the pooled library, 5 μl aliquots from each normalized sample (vaginal, n=272; fecal, n=64) were combined. The exact size of library products and the library concentration were measured with a KAPA Library Quantification Kit (Kapa Biosystems, Woburn, Mass., USA) through quantitative PCR (qPCR, Eppendorf, Westbury, N.Y., USA) and an Agilent 2100 Bioanalyzer System (Agilent, Santa Clara, Calif., USA). The library was diluted based on the qPCR and bioanalyzer results²⁴.

The 20 nM pooled library, containing 336 individual samples, and a PhiX control v3 (20 nM) (Illumina) were mixed with 0.2 N NaOH and HT1 buffer (Illumina). PhiX control v3 (5%, v/v) (Illumina) was added to the mix and 600 μl were loaded into a MiSeq® v2 (500 cycle) reagent cartridge for sequencing. The sequencing procedure was monitored periodically throughout the assay using the Illumina BaseSpace® website. The raw sequence files of all 336 samples were submitted to the National Center for Biotechnology Information (NCBI) Short Read Archive database and are available under BioProject accession number PRJNA 497069.

Sequence Analysis:

The demultiplexed R1 and R2 sequencing read files (approximately 250 base pairs in length) were downloaded to a local computer from the Illumina BaseSpace® website and the data were processed using the DEBLUR program integrated in the QIIME2 pipefine^{26,27}. The Uchime algorithm was used to remove chimeric sequences²⁸. Sequences were considered to be high quality if more than 90% of the bases had Phred scores greater than 30 and they passed the error reducing, chimera detection, and removal steps. The sequences were assigned to features using a 100% cutoff. These features were classified using the naive Bayes method²⁹ and the Greengenes (13_8 clustered at 99% similarity) database was used for the training of the 16S Classifier. The number of reads were subsampled to 3,000 and 1,000 for fecal samples and vaginal swabs, respectively, to reduce sequencing bias before downstream analysis.

Ecological and Statistical Analyses:

For all analyses, significance was determined as P<0.05. Alpha diversity, Shannon diversity index³⁰, and richness (number of observed operational taxonomic units (OTUs)) were calculated using QIIME2. The Kruskal-Wallis test was performed to identify differences in alpha diversity (Shannon Diversity index and richness) in fecal and vaginal samples between heifers who established a pregnancy and those that did not. Examined variables included breeding method, health status of the calf at calving, and stage of pregnancy. Beta diversity was evaluated using Bray-Curtis³¹ and Jaccard³² distances calculated in QIIME2, which explore the dissimilarity between the communities' structure and membership, respectively. Random forest was used to identify and rank microbial signatures that accurately differentiate groups of female cattle. This machine learning technique accounts for non-linear relationships and dependencies among all microbial features. The relative abundance of the top 1,500 features and alpha-diversity measures were included as inputs for the random forest model. Each input (feature) was given an importance score (MDA: mean decrease accuracy) based on the increase in error caused by removing that feature from the predictors.

### Results:

### Sequencing Depth and Alpha Diversities

Prior to breeding and during each trimester of gestation, 336 samples were collected from commercial beef heifers (vaginal: n=272, fecal: n=64). DNA extraction and bar-coded pyrosequencing of the V4 region of the 16S rRNA gene was performed on each sample. After removing low quality reads and chimeras using qiime2 (2018.8), 3,617,919 and 1,584,626 high quality reads remained for vaginal and fecal samples respectively. Vaginal samples averaged 13,862 reads per sample, ranging from 1,153 to 98,623 reads. Fecal samples averaged 26,410 reads per sample, ranging from 3,045 to 98,623 reads. These sequences were assigned to 9,496 features from vaginal samples and 4,696 features from fecal samples based on 100% sequence similarity. Sequence number was normalized to 1,000 for vaginal samples and to 3,000 for fecal samples to standardize sampling for downstream alpha and beta diversity analyses.

TABLE 1
P values related to alpha diversity measures in vaginal samples based on pregnancy status.¹

| | | | Change in | | | | | Change in | |
|---|---|---|---|---|---|---|---|---|---|
| | Comparison | | Diversity | P value | | Comparison | | Diversity | P value |
| Shannon | 1 Bred | 1 Open | | 0.243 | Observed | 1 Bred | 1 Open | | 0.158 |
| Index | 2 Bred | 2 Open | | 0.445 | OTUs | 2 Bred | 2 Open | | 0.962 |
| | 3 Bred | 3 Open | | 0.626 | | 3 Bred | 3 Open | | 0.437 |
| | 1 Bred | 2 Bred | | 0.119 | | 1 Bred | 2 Bred | | 0.625 |
| | 1 Bred | 3 Bred | Increase | 0.0005* | | 1 Bred | 3 Bred | Increase | 0.002* |
| | 1 Bred | 4 Bred | | 0.146 | | 1 Bred | 4 Bred | | 0.876 |
| | 1 Open | 2 Open | | 0.140 | | 1 Open | 2 Open | | 0.110 |
| | 1 Open | 3 Open | Increase | 0.001* | | 1 Open | 3 Open | Increase | 0.002* |
| | 2 Bred | 3 Bred | Increase | 0.018* | | 2 Bred | 3 Bred | Increase | 0.003* |
| | 2 Bred | 4 Bred | | 0.935 | | 2 Bred | 4 Bred | | 0.419 |
| | 2 Open | 3 Open | Increase | 0.033* | | 2 Open | 3 Open | Increase | 0.046 |
| | 3 Bred | 4 Bred | Decrease | 0.047* | | 3 Bred | 4 Bred | Decrease | 0.001* |

¹Vaginal samples were obtained from 72 beef heifers. Individuals that established pregnancy (n = 56) were sampled before breeding (stage 1) and at three time points during gestation (stages 2, 3, and 4). Individuals that failed to establish pregnancy (n = 16) were sampled before breeding (stage 1) and during the first and second trimesters of gestation (stages 2 and 3).
*Pair-wise comparisons between stage and pregnancy status were determined to be statistically significant at P < 0.05.

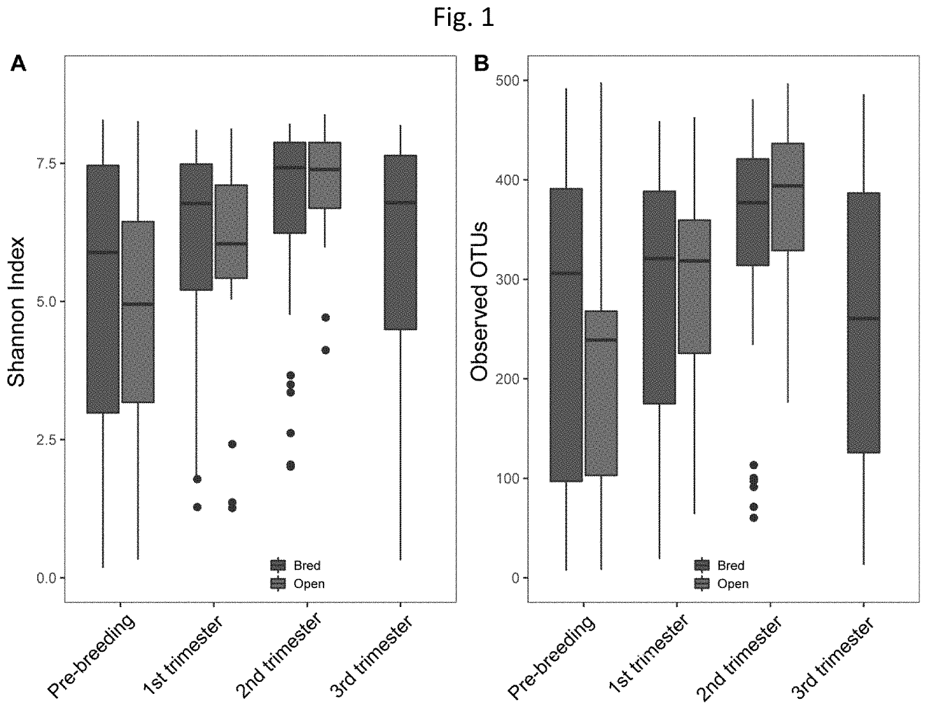

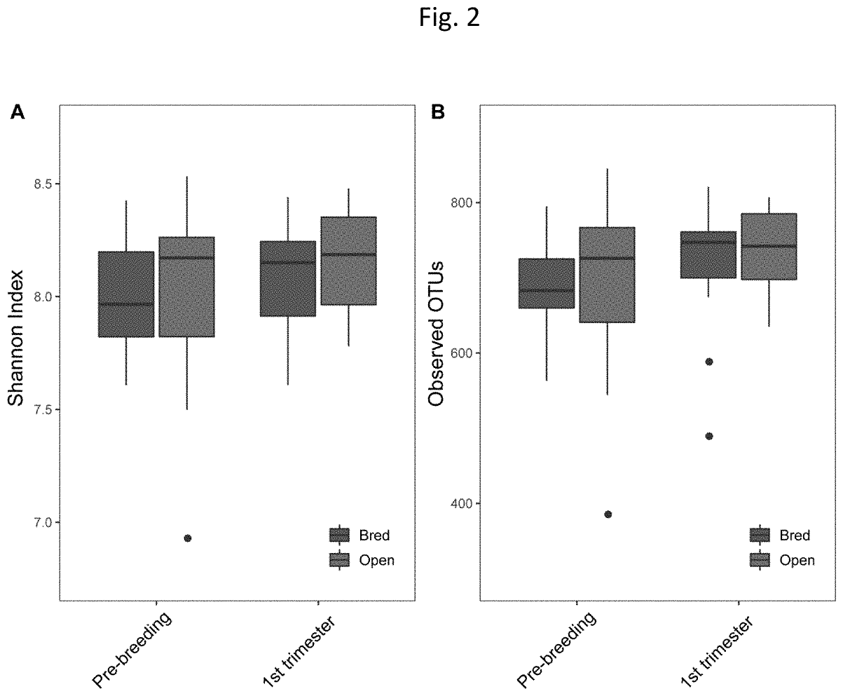

At the community level, significant differences in alpha diversity indices (Shannon index and the number of observed operational taxonomic units (OTUs)) were observed over time in the vaginal microbiome (FIG. 1A, Kruskal-Wallis test, P=6.475e-05, FIG. 1B, Kruskal-Wallis test, P=3.149e-05). In animals both with and without established pregnancies, microbial diversity (Shannon index) increased from pre-breeding to the second trimester (P<0.05, Table 1) and from the first trimester to the second trimester (P<0.05, Table 1), but then decreased from the second trimester to the third trimester (P<0.05, Table 1). Both open and bred individuals showed an increase in the number of observed OTUs (an indication of community richness) from pre-breeding to the second trimester (P<0.05, Table 1) and from the first trimester to the second trimester (P<0.05, Table 1). However, the number of observed OTUs decreased in bred females from the second trimester to the third trimester (P<0.05, Table 1). For fecal samples, we did not detect any significant differences in Shannon indices over time or between animals of different pregnancy statuses (FIG. 2A, Kruskal-Wallis test, P=0.53). Consistently, no significant differences in the total number of observed OTUs were found in fecal samples (FIG. 2B, Kruskal-Wallis test, P=0.24). P values for pairwise comparison of fecal samples from bred and open cattle are presented in Table 2. No significant differences in fecal or vaginal alpha diversity measures (Kruskal-Wallis, fecal: p=0.59; vaginal: p=0.155) were observed between the open and the bred female cattle at any time point.

TABLE 2
P values related to alpha diversity measures
in fecal samples based on pregnancy status.¹

| | Comparison | | P value | | Comparison | | P value |
|---|---|---|---|---|---|---|---|
| Shannon | 1 Bred | 1 Open | 0.4945 | Observed | 1 Bred | 1 Open | 0.6073 |
| Index | 2 Bred | 2 Open | 0.3519 | OTUs | 2 Bred | 2 Open | 0.8361 |
| | 1 Bred | 2 Bred | 0.4773 | | 1 Bred | 2 Bred | 0.085 |
| | 1 Open | 2 Open | 0.5249 | | 1 Open | 2 Open | 0.3084 |

¹Fecal samples were obtained from 32 beef heifers. Individuals that established a pregnancy (n = 16) and those that did not (n = 16) were sampled before breeding (stage 1) and during the first trimester (stage 2).
*Pair-wise comparisons between stage and pregnancy status were determined to be statistically significant at P < 0.05.

### Community Membership and Structure

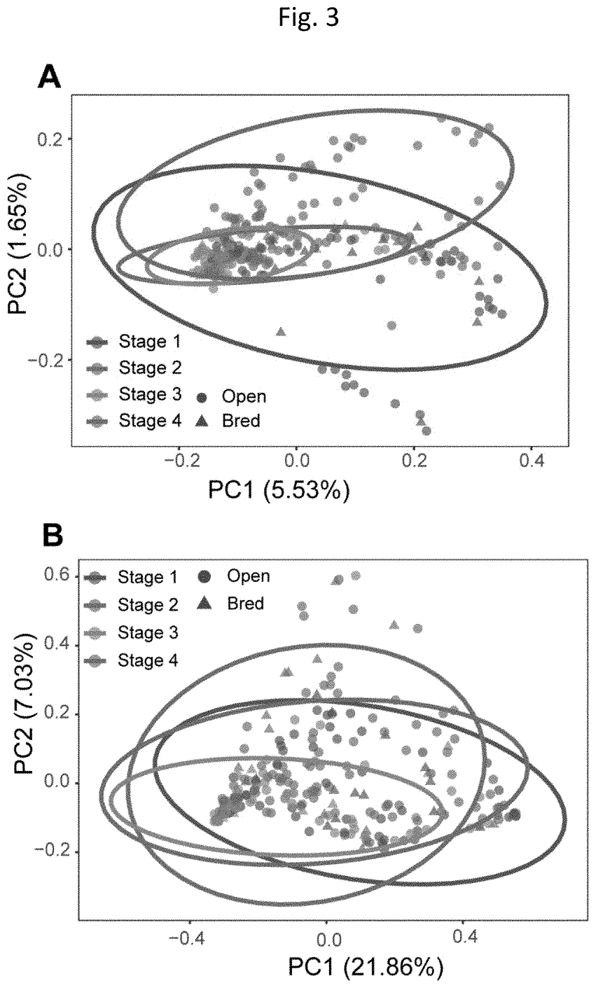

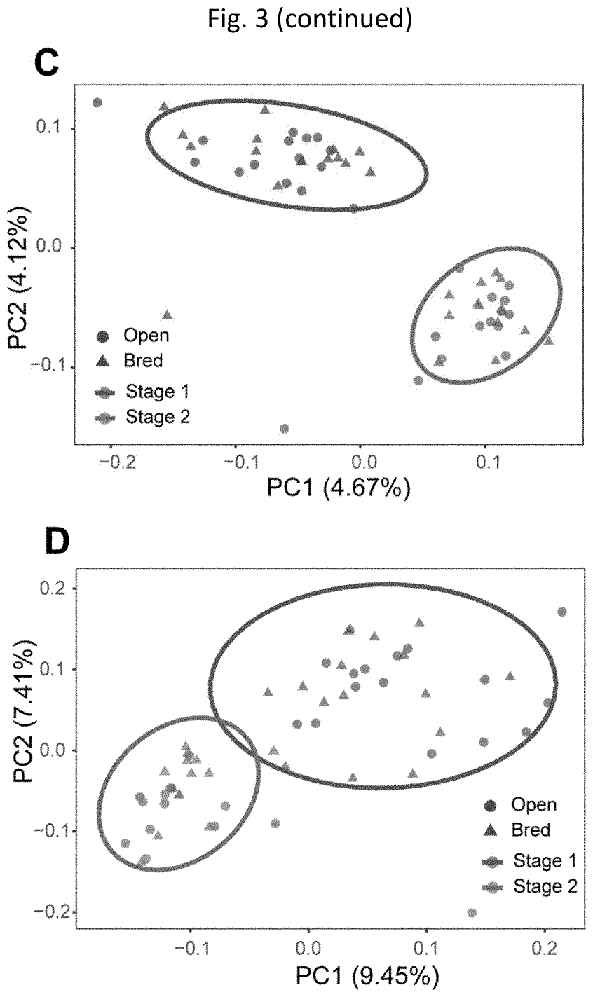

We next compared community membership and structure between pregnant and non-pregnant females over time. To evaluate bacterial community membership, principal coordinate analysis (PCoA) was applied to the Jaccard dissimilarity matrix. Vaginal samples representing each time point and each pregnancy status cluster together on principle coordinate axes 1 and 2 in these plots (PC1, PC2; FIG. 3A). While no differences associated with pregnancy status were detected (Analysis of similarity, ANOSIM, stage 1: P=0.542, R=−0.018; stage 2: P=0.805, R=−0.075; stage 3: P=0.856, R=−0.099), differences in community membership were observed over time (ANOSIM, R=0.147, P<0.05). The Bray-Curtis index was used to estimate dissimilarities in both community membership and structure. A PCoA plot based on Bray-Curtis distance shows no distinct clustering according to pregnancy status or time (FIG. 3B). While no differences associated with pregnancy status were observed (stage 1: P=0.452, R=0.008; stage 2: P=0.673, R=−0.029; stage 3: P=0.825, R=−0.063), differences in community structure were observed over time (ANOSIM, R=0.138, P<0.05).

Interestingly, a PCoA plot based on Jaccard distance for fecal samples shows distinct clustering patterns over time (FIG. 3C, ANOSIM, R=0.391, P<0.001), though no differences based on pregnancy status were observed (stage 1: P=0.354, R=0.011; stage 2: P=0.418, R=0.007). Consistently, significant changes in fecal community structure over time are also shown in the PCoA plot based on Bray-Curtis distances (FIG. 3D). No differences based on pregnancy status were seen (stage 1: P=0.4, R=0.006; stage 2: P=0.789, R=−0.029).

### Community Composition

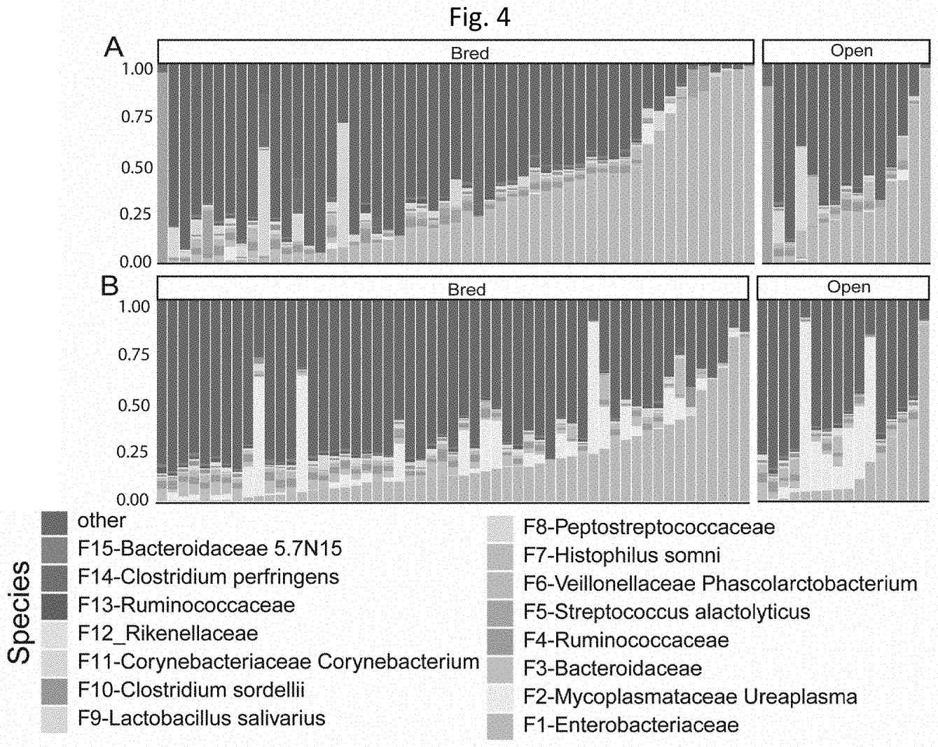

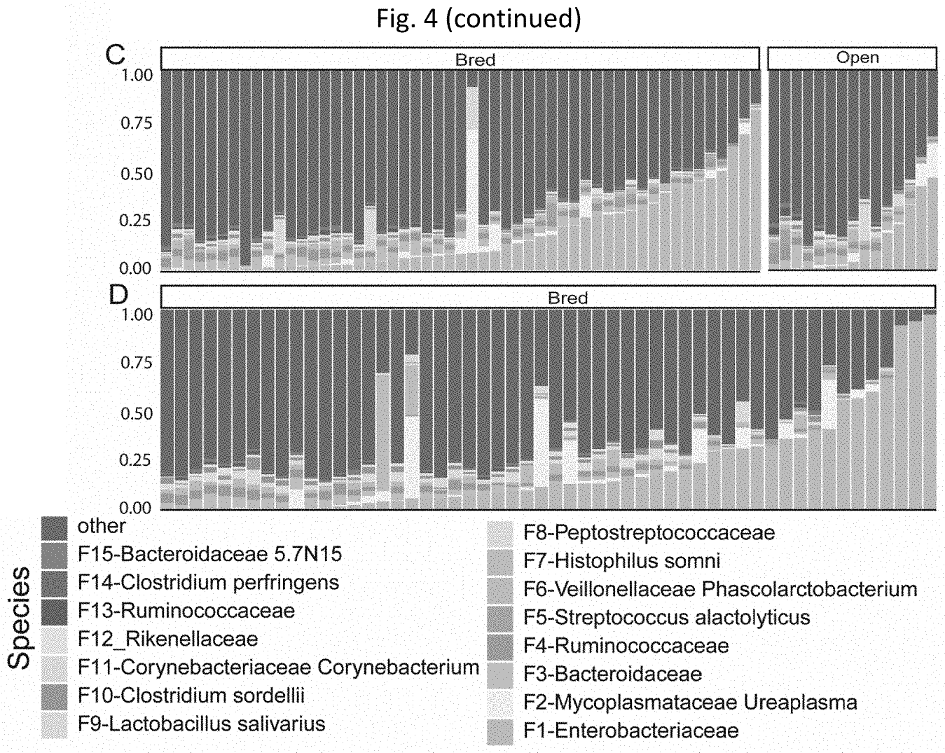

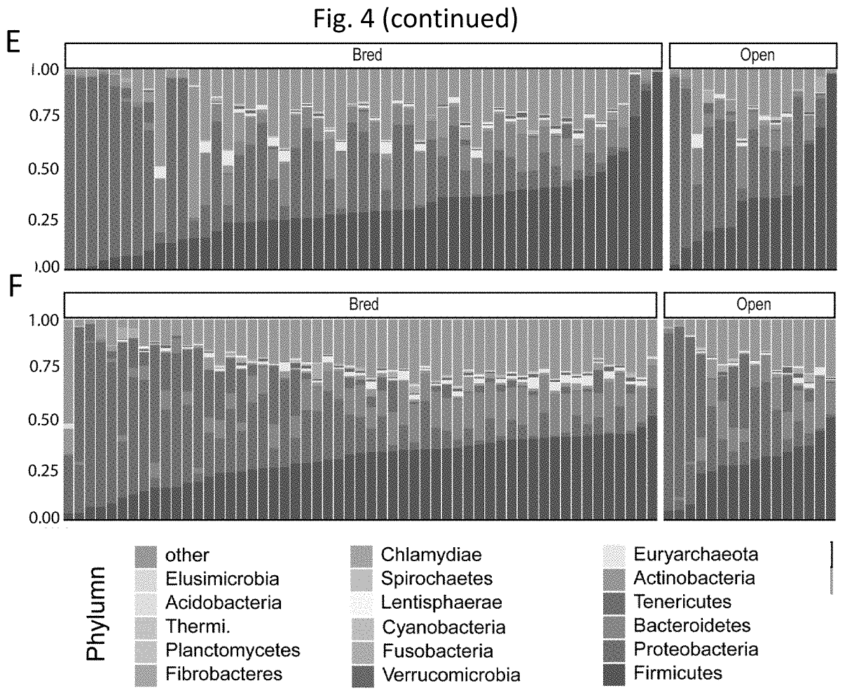

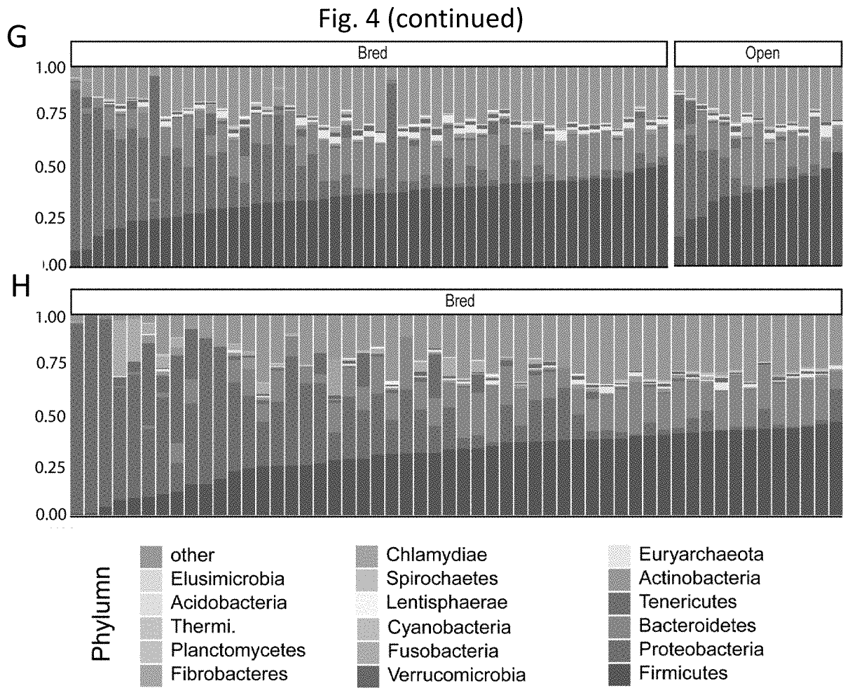

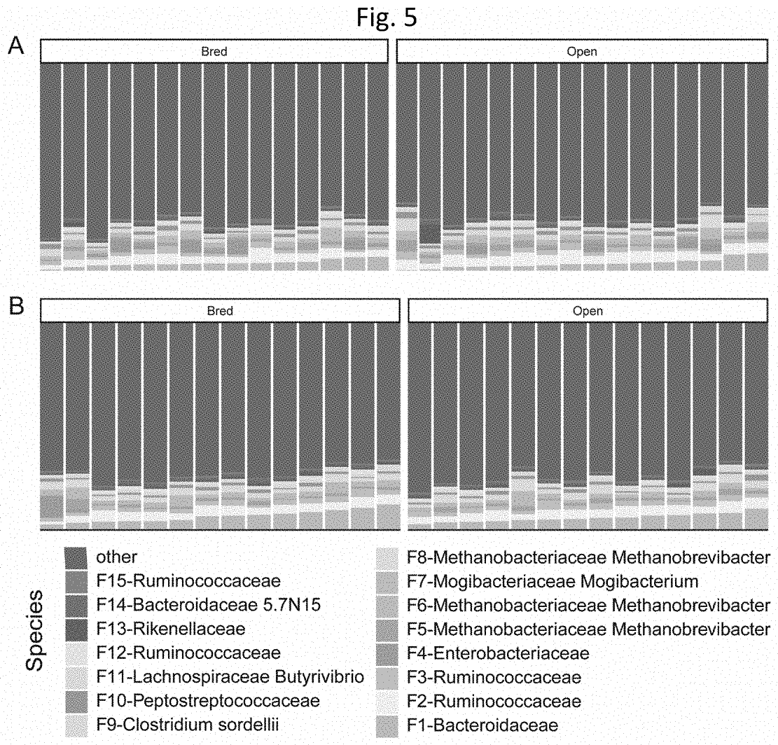

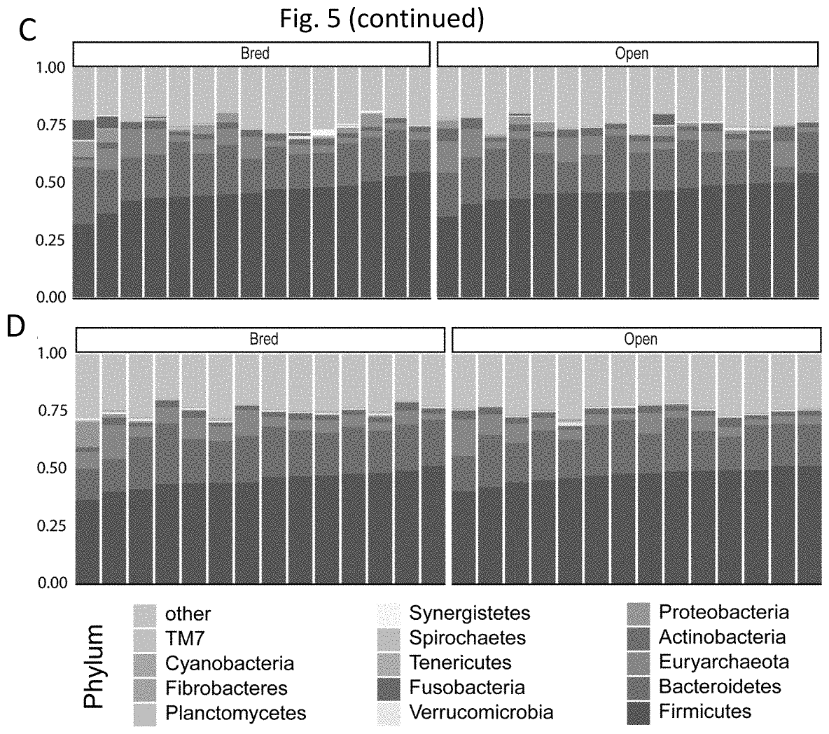

The top 15 most abundant bacterial features of the bovine vaginal microbiome are shown in FIG. 4A-4D. The vaginal microbiome is dominated by unclassified Enterobacteriaceae (21.05%), followed by Ureaplasma (4.37%) and unclassified Bacteroidaceae (2.49%, FIG. 4A-4D). At the phylum level, Firmicutes is the most dominant taxon (31.57%), followed by Proteobacteria (24.08%), Bacteroidetes (12.96%), and Tenericutes (4.95%, FIG. 4E-411). These four phyla constitute 79.30% of the bacteria in the bovine vaginal microbiome. In the fecal microbiome, the top 15 most abundant features include several features associated with Ruminococcaceae and Bacteroidaceae (FIG. 5A-5B). At the phylum level, Firmicutes (45.93%), Bacteroidetes (18.83%), Euryarchaeota (6.14%) and Actinobacteria (2.57%) were the most abundant taxa, constituting 73.47% of the fecal bacterial community (FIG. 5C-5D).

### Bacterial Features Predictive of Pregnancy Status

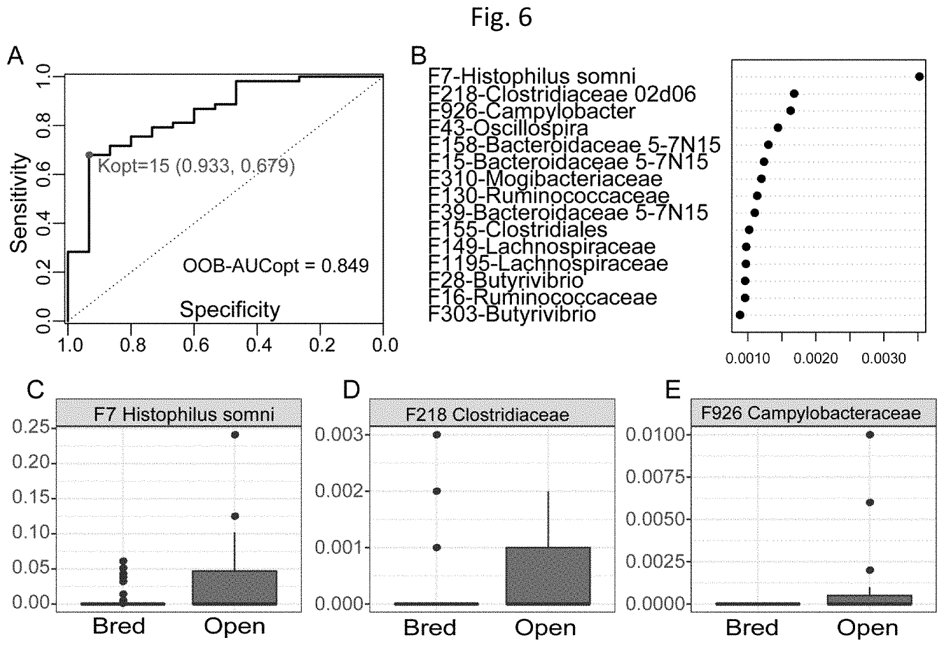

To assess whether a pre-breeding sample of the vaginal or fecal microbiome could be used to predict successful pregnancy, we developed a random forest model to identify the bacterial features that are most predictive of pregnancy status. We determined the optimal model based on the maximum area under the curve (AUC) using the AUC-RF algorithm. For the vaginal microbiome, 15 vaginal features selected by random forest were able to predict if a cow can get pregnant with an AUC of 0.849 (sensitivity 0.933, specificity 0.679, FIG. 6A, Table 3). The top three vaginal features, including Histophilus somni, Clostridiaceae 02d06, and Campylobacter, were all more abundant in the open cows (FIG. 6B-6E).

TABLE 3
Selected features from the vaginal microbiome

| Partial 16S | |
|---|---|
| rRNA sequence | Source strain |
| SEQ ID NO: 1 | Histophilus somni strain 8025 |
| SEQ ID NO: 2 | Colidextribacter massiliensis strain Marseille-P3083 |
| SEQ ID NO: 3 | Campylobacter lanienae strain CCUG 44467 |
| SEQ ID NO: 4 | Oscillibacter ruminantium strain GH1 |
| SEQ ID NO: 5 | Bacteroides plebeius strain M12 |
| SEQ ID NO: 6 | Bacteroides plebeius strain M12 |
| SEQ ID NO: 7 | Ihubacter massiliensis strain Marseille-P2843 |
| SEQ ID NO: 8 | Colidextribacter massiliensis strain Marseille-P3083 |
| SEQ ID NO: 9 | Bacteroides plebeius strain M12 |
| SEQ ID NO: 10 | Intestinimonas butyriciproducens strain SRB-521-5-1 |
| SEQ ID NO: 11 | Bacteroides xylanolyticus strain X5-1 |
| SEQ ID NO: 12 | Merdimonas faecis strain BR31 |
| SEQ ID NO: 13 | Ruminococcus lactaris strain ATCC 29176 |
| SEQ ID NO: 14 | [Clostridium] cellobioparum strain DSM 1351 |
| SEQ ID NO: 15 | Lactonifactor longoviformis strain ED-Mt61/PYG-s6 |

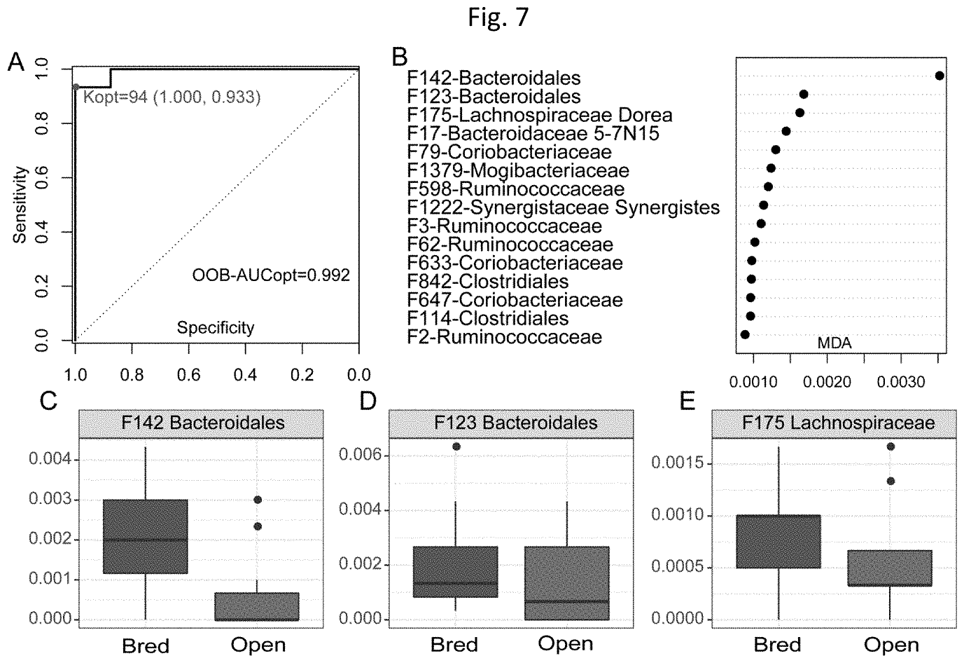

Surprisingly, the pre-breeding fecal microbiome predicted the capability of a cow to establish pregnancy after breeding with even higher accuracy (AUC=0.992, sensitivity=1.0, specificity=0.933, FIG. 7A, Table 4). Although 93 features were needed to obtain such a high accuracy, the top 15 features (FIG. 7B) alone yielded a very high AUC (0.929). The relative abundance and distribution of the top three features between open and bred fecal samples are shown in FIG. 7C-7E. All three features (two associated with Bacteroidales and one with Lachnospiraceae) were more abundant in the feces of cows that established pregnancy after breeding.

TABLE 4
Selected features from the fecal microbiome

| Partial 16S | |
|---|---|
| rRNA sequence | Source strain |
| SEQ ID NO: 16 | Parapedobacter lycopersici strain T16R-256 |
| SEQ ID NO: 17 | Parapedobacter soli strain DCY14 |
| SEQ ID NO: 18 | [Clostridium] hylemonae strain TN-271 |
| SEQ ID NO: 19 | Bacteroides mediterraneensis strain Marseille-P2644 |
| SEQ ID NO: 20 | Enterorhabdus muris strain WCA-131-CoC-2 |
| SEQ ID NO: 21 | Eubacterium pyruvativorans strain 1-6 |
| SEQ ID NO: 22 | Monoglobus pectinilyticus strain 14 |
| SEQ ID NO: 23 | Cloacibacillus porcorum strain CL-84 |
| SEQ ID NO: 24 | Monoglobus pectinilyticus strain 14 |
| SEQ ID NO: 25 | Harryflintia acetispora strain V20-281a |
| SEQ ID NO: 26 | Collinsella massiliensis strain GD3 |
| SEQ ID NO: 27 | Monoglobus pectinilyticus strain 14 |
| SEQ ID NO: 28 | Denitrobacterium detoxificans strain NPOH1 |
| SEQ ID NO: 29 | Novibacillus thermophilus strain SG-1 |
| SEQ ID NO: 30 | Monoglobus pectinilyticus strain 14 |

### DISCUSSION

In contrast to what has been reported in humans^{35,36}, no significant differences in evenness or richness were observed in the bacterial communities of bred and open heifers based on vaginal and fecal samples. Studies suggest that humans develop more stable vaginal microbiota near the end of the gestation period. For example, Aagaard et al³⁵ reported decreased species richness and diversity that progressed with gestational age. Interestingly, significant differences in bovine fecal microbial membership and structure were detected over time, though such changes could be due to a variety of drivers (e.g., environment, diet, hormones). However, no change in community membership or structure was observed in the bovine vaginal niche throughout gestation, suggesting that this microbiome is stable and not affected by any of these factors.

To improve bovine reproduction strategies, it is of particular interest to be able to predict the likelihood of a heifer to establish a pregnancy. Using random forest, we identified 15 bacterial features within the vaginal microbiome that accurately (AUC=0.849) differentiate heifers that established pregnancy from those that never did at the pre-breeding stage. A feature associated with Histophilus somni was listed as the top predictor of pregnancy. Rodrigues and colleagues¹⁷ identified Histophilus as one of three genera that dominate the vaginal microflora of heifers with reproductive disorder. Histophilus are gram-negative, non-spore-forming bacteria that exist in both pathogenic and non-pathogenic forms, both of which can be isolated from the bovine vagina⁴⁴. The increased abundance of Histophilus in vaginal samples from females that do not establish a pregnancy is consistent with the association of this bacterium with reproductive disorder. The #2 predictor of pregnancy was a feature associated with Clostridiaceae. Certain species of Clostridiaceae have been linked with bacterial vaginosis in humans^{20,46}, but little is known about the role of Clostridiaceae in animal reproduction. The #3 predictor of pregnancy was a feature associated with Campylobacter. Campylobacter can cause bovine venereal campylobacteriosis or vibriosis, which is the primary cause of abortion and infertility in cattle^{47,48}. Consistent with this link to infertility, this bacterium was only detected in female cattle that did not establish a pregnancy in the current study.

Surprisingly, the bovine fecal microbiome could be used to predict the establishment of pregnancy with even higher accuracy than the vaginal microbiome (AUC=0.929), even using just 15 bacterial features. These features are associated with Bacteroidales, Ruminococcaceae, and Coriobacteriaceae. Coriobacteriaceae has been isolated from the vagina of cattle with and without reproductive disorder, but it is known for its symbiotic role in the gut of insects^{17,50}. This gram-positive, obligate anaerobe works to ferment glucose and other compounds found in the food of insects, producing lactic acid, ethanol, CO₂ and H₂⁵⁰. Three members of Coriobacteriaceae were listed in the top 15 predictors of pregnancy in the feces. The relative abundance of Coriobacteriaceae is lower in cattle that become pregnant than in those that never establish pregnancy

### REFERENCES

1 Hess, B. et al. Nutritional controls of beef cow reproduction. Journal of Animal Science 83, E90-E106 (2005).
2 Bellows, D., Ott, S. & Bellows, R. Cost of Reproductive Diseases and Conditions in Cattle 1. The Professional Animal Scientist 18, 26-32 (2002).
3 Lamb, G. C. et al. Invited Review: Advantages of current and future reproductive technologies for beef cattle production 1, 2. Professional Animal Scientist 32, 162-171 (2016).
4 Dziuk, P. & Bellows, R. Management of reproduction of beef cattle, sheep and pigs. Journal of Animal Science 57, 355-379 (1983).
5 Schillo, K. K., Hall, J. B. & Hileman, S. M. Effects of nutrition and season on the onset of puberty in the beef heifer. Journal of Animal Science 70, 3994 (1992).
6 Zhou, X. et al. Differences in the composition of vaginal microbial communities found in healthy Caucasian and black women. Isme Journal 1, 121-133 (2007).
7 Gajer, P. et al. Temporal dynamics of the human vaginal microbiota. Science translational medicine 4, 132ra152-132ra152 (2012).
8 Ravel, J. et al. Vaginal microbiome of reproductive-age women. Proceedings of the National Academy of Sciences 108, 4680-4687 (2011).
9 Verstraelen, H. et al. Longitudinal analysis of the vaginal microflora in pregnancy suggests that L. crispatus promotes the stability of the normal vaginal microflora and that L. gasseri and/or L. iners are more conducive to the occurrence of abnormal vaginal microflora. BMC microbiology 9, 116 (2009).
10 Boris, S. & Barbés, C. Role played by lactobacilli in controlling the population of vaginal pathogens. Microbes and infection 2, 543-546 (2000).
11 Sobel, J. D. Bacterial vaginosis. Annual review of medicine 51, 349-356 (2000).
12 Koumans, E. H. et al. The prevalence of bacterial vaginosis in the United States, 2001-2004; associations with symptoms, sexual behaviors, and reproductive health. Sexually Transmitted Diseases 34, 864-869 (2007).
13 Salah, R. M., Allam, A. M., Magdy, A. M. & Mohamed, A. S. Bacterial vaginosis and infertility: cause or association? European journal of obstetrics & gynecology and reproductive biology 167, 59-63 (2013).
14 Yildirim, S. et al. Primate vaginal microbiomes exhibit species specificity without universal Lactobacillus dominance. The ISME journal 8, 2431 (2014).
15 Swartz, J. D. et al. Characterization of the vaginal microbiota of ewes and cows reveals a unique microbiota with low levels of lactobacilli and near-neutral pH. Frontiers in veterinary science 1, 19 (2014).
16 Gonzalez Moreno, C., Fontana, C., Cocconcelli, P. S., Callegari, M. L. & Otero, M. C. Vaginal microbial communities from synchronized heifers and cows with reproductive disorders. Journal of applied microbiology 121, 1232-1241 (2016).
17 Rodrigues, N. et al. Qualitative analysis of the vaginal microbiota of healthy cattle and cattle with genital-tract. Genetics and Molecular Research 14, 6518-6528 (2015).
18 Ott, S. et al. Reduction in diversity of the colonic mucosa associated bacterial microflora in patients with active inflammatory bowel disease. Gut 53, 685-693 (2004).
19 Manichanh, C. et al. Reduced diversity of faecal microbiota in Crohn's disease revealed by a metagenomic approach. Gut 55, 205-211, doi:10.1136/gut.2005.073817 (2006).
20 Fredricks, D. N., Fiedler, T. L. & Marrazzo, J. M. Molecular identification of bacteria associated with bacterial vaginosis. New England Journal of Medicine 353, 1899-1911 (2005).
21 Othman, M., Alfirevic, Z. & Neilson, J. P. Probiotics for preventing preterm labour. Cochrane Database of Systematic Reviews (2007).
22 Gerritsen, J., Smidt, H., Rijkers, G. T. & Vos, W. M. Intestinal microbiota in human health and disease: the impact of probiotics. Genes & nutrition 6, 209 (2011).
23 Shreiner, A. B., Kao, J. Y. & Young, V. B. The gut microbiome in health and in disease. Current opinion in gastroenterology 31, 69 (2015).
24 McClure, M. W. The Vaginal Microbiome Related to Reproductive Traits in Beef Heifers. (2018).
25 Kozich, J. J., Westcott, S. L., Baxter, N. T., Highlander, S. K. & Schloss, P. D. Development of a Dual-Index Sequencing Strategy and Curation Pipeline for Analyzing Amplicon Sequence Data on the MiSeq Illumina Sequencing Platform. Applied & Environmental Microbiology 79, 5112 (2013).
26 Amir, A. et al. Deblur rapidly resolves single-nucleotide community sequence patterns. MSystems 2, e00191-00116 (2017).
27 Bolyen, E. et al. QIIME 2: Reproducible, interactive, scalable, and extensible microbiome data science. Report No. 2167-9843, (PeerJ Preprints, 2018).
28 Edgar, R. C., Haas, B. J., Clemente, J. C., Quince, C. & Knight, R. UCHIME improves sensitivity and speed of chimera detection. Bioinformatics 27, 2194-2200 (2011).
29 Pedregosa, F. et al. Scikit-learn: Machine learning in Python. Journal of machine learning research 12, 2825-2830 (2011).
30 Shannon, C. E. The Mathematical Theory of Communication. By C E Shannon and Warren Weaver. (Urbana, 1949).
31 Bray, J. R. & Curtis, J. T. An ordination of the upland forest communities of southern Wisconsin. Ecological monographs 27, 325-349 (1957).
32 Chao, A., Chazdon, R. L., Colwell, R. K. & Shen, T. J. A new statistical approach for assessing similarity of species composition with incidence and abundance data. Ecology letters 8, 148-159 (2005).
33 Otero, C. et al. Vaginal bacterial microflora modifications during the growth of healthy cows. Letters in applied microbiology 31, 251-254 (2000).
34 Nader-Macias, M. E. F., Otero, M. C., Espeche, M. C. & Maldonado, N. C. Advances in the design of probiotic products for the prevention of major diseases in dairy cattle. Journal of industrial microbiology & biotechnology 35, 1387-1395 (2008).
35 Aagaard, K. et al. A metagenomic approach to characterization of the vaginal microbiome signature in pregnancy. PloS one 7, e36466 (2012).
36 Oakley, B. B., Fiedler, T. L., Marrazzo, J. M. & Fredricks, D. N. Diversity of human vaginal bacterial communities and associations with clinically defined bacterial vaginosis. Applied and environmental microbiology 74, 4898-4909 (2008).
37 Smith, S. B. & Ravel, J. The vaginal microbiota, host defence and reproductive physiology. The Journal of physiology 595, 451-463 (2016).
38 Sheldon, I. Genes and environmental factors that influence disease resistance to microbes in the female reproductive tract of dairy cattle. Reproduction, Fertility and Development 27, 72-81 (2015).
39 Williams, E. J. et al. Clinical evaluation of postpartum vaginal mucus reflects uterine bacterial infection and the immune response in cattle. Theriogenology 63, 102-117 (2005).
40 Gilbert, R. O., Shin, S. T., Guard, C. L., Erb, H. N. & Frajblat, M. Prevalence of endometritis and its effects on reproductive performance of dairy cows. Theriogenology 64, 1879-1888 (2005).
41 Mulira, G. L., Saunders, J. R. & Barth, A. D. Isolation of Ureaplasma diversum and mycoplasmas from genital tracts of beef and dairy cattle in Saskatchewan. The Canadian Veterinary Journal 33, 46 (1992).
42 Ruhnke, H., Doig, P., MacKay, A., Gagnon, A. & Kierstead, M. Isolation of Ureaplasma from bovine granular vulvitis. Canadian Journal of Comparative Medicine 42, 151 (1978).
43 Thomas, A. et al. Isolation of mycoplasma species from the lower respiratory tract of healthy cattle and cattle with respiratory disease in Belgium. The Veterinary Record 151, 472 (2002).
44 Janzen, E. Overview of Histophilosis. Merck Veterinary Manual [online] Available at: http://www.merckmanuals.com/vet/generalized_conditions/histophilosis/overview_of_histophilosis.html [Accessed Mar. 20 2018] (2018).
45 Clemmons, B. A. et al. Vaginal and uterine bacterial communities in postpartum lactating cows. Frontiers in microbiology 8, 1047 (2017).
46 Dareng, E. et al. Prevalent high-risk HPV infection and vaginal microbiota in Nigerian women. Epidemiology & Infection 144, 123-137 (2016).
47 Givens, M. D. A clinical, evidence-based approach to infectious causes of infertility in beef cattle. Theriogenology 66, 648-654 (2006).
48 Hoffer, M. Bovine campylobacteriosis: a review. The Canadian Veterinary Journal 22, 327 (1981).
49 SALEH, M., HARKINEZHAD, M. & SALMANI, V. Detection of some bacterial causes of abortion in Afshari sheep using Real Time PCR detection and sensitivity assessment of Campylobacter primers. (2014).
50 HAAS, F. & KÖNIG, H. Coriobacterium glomerans gen. nov., sp. nov. from the intestinal tract of the red soldier bug. International Journal of Systematic and Evolutionary Microbiology 38, 382-384 (1988).
51 Timsit, E. et al. Evolution of the nasopharyngeal microbiota of beef cattle from weaning to 40 days after arrival at a feedlot. Veterinary microbiology 187, 75-81 (2016).
52 Mu, Y. et al. High-production dairy cattle exhibit different rumen and fecal bacterial community and rumen metabolite profile than low-production cattle. Microbiology Open, e00673 (2018).
53 Young, W., Hine, B. C., Wallace, 0. A., Callaghan, M. & Bibiloni, R. Transfer of intestinal bacterial components to mammary secretions in the cow. Peer J 3, e888 (2015).
54 Rodrigues, M., Lima, S., Canniatti-Brazaca, S. & Bicalho, R. The microbiome of bulk tank milk: characterization and associations with somatic cell count and bacterial count. Journal of dairy science 100, 2536-2552 (2017).

What is claimed:

1. A method for selecting female cows to include in a breeding program, the method comprising:
collecting a vaginal swab sample from a female cow;
measuring the level of at least one biomarker associated a bacterium of a species from the group consisting of: Histophilus somni, Colidextribacter massiliensis, Campylobacter lanienae, Bacteroides plebeius, Bacteroides xylanolyticus, Ihubacter massiliensis, Intestinimonas butyriciproducens, Merdimonas faecis, Ruminococcus lactaris, Lactonifactor longoviformis, Oscillibacter ruminantium, and [Clostridium] cellobioparum;
and analyzing the abundance of the biomarker to determine whether to breed the female cow.

2. The method of claim 1, wherein the female cow is bred if one or more of the following differences in the abundance of a biomarker associated with a bacterial species is detected: a decrease in Histophilus somni, decrease in Colidextribacter massiliensis, decrease in Campylobacter lanienae, decrease in Bacteroides xylanolyticus, decrease in Ihubacter massiliensis, decrease in Intestinimonas butyriciproducens, decrease in Merdimonas faecis, decrease in Ruminococcus lactaris, decrease in Lactonifactor longoviformis, increase in Oscillibacter ruminantium, or an increase in [Clostridium] cellobioparum.

3. The method of claim 1, wherein the measured biomarker is associated with a bacterium of one or more of following strains: Histophilus somni strain 8025, Colidextribacter massiliensis strain Marseille-P3083, Campylobacter lanienae strain CCUG, Oscillibacter ruminantium strain GH1, Bacteroides plebeius strain M12, Ihubacter massiliensis strain Marseille, Intestinimonas butyriciproducens strain SRB-521-5-I, Bacteroides xylanolyticus strain X5-1, Merdimonas faecis strain BR31, Ruminococcus lactaris strain ATCC, [Clostridium] cellobioparum strain DSM 1351, or Lactonifactor longoviformis strain ED-Mt61/PYG-s6.

4. The method of claim 1, wherein the measured biomarker is a biomarker associated with a bacterium selected from the group consisting of Campylobacter, Merdimonas and Lactonifactor, and wherein the female cow is bred if the biomarker is not detected.

5. The method of claim 1, wherein the step of measuring the level of a biomarker comprises:
a) detecting a protein associated with a particular bacterium; or
b) detecting a nucleic acid associated with a particular bacterium.

6. The method of claim 5, wherein the nucleic acid is a component of a 16S or 23S ribosomal subunit.

7. The method of claim 6, wherein the nucleic acid comprises a sequence selected from the group consisting of SEQ ID NOs: 1-30.

8. The method claim 1, wherein the sample is collected from a female cow prior to estrus synchronization, during estrus synchronization, prior to the onset of estrus, or prior to breeding.

9. A method for selecting female cows to include in a breeding program, the method comprising:
collecting a fecal sample from a female cow;
measuring the level of at least one biomarker associated a bacterium of a species from the group consisting of: Bacteroides mediterraneensis, Enterorhabdus muris, Eubacterium pyruvativorans, Monoglobus pectinilyticus, Harryflintia acetispora, Collinsella massiliensis, Denitrobacterium detoxificans, Parapedobacter lycopersici, Parapedobacter soli, [Clostridium] hylemonae, Cloacibacillus porcorum, and Novibacillus thermophiles;
and analyzing the abundance of the biomarker to determine whether to breed the female cow.

10. The method of claim 9, wherein the female cow is bred if one or more of the following differences in the abundance of a biomarker associated with a bacterial species is detected: a decrease in Bacteroides mediterraneensis, decrease in Enterorhabdus muris, decrease in Eubacterium pyruvativorans, decrease in Harryflintia acetispora, decrease in Collinsella massiliensis, decrease in Denitrobacterium detoxificans, increase in Parapedobacter lycopersici, increase in Parapedobacter soli, increase in [Clostridium] hylemonae, increase in Cloacibacillus porcorum, or an increase in Novibacillus thermophiles.

11. The method of claim 9, wherein the measured biomarker is associated with a bacterium of one or more of following strains: Parapedobacter lycopersici strain T16R-256, Parapedobacter soli strain DCY14, [Clostridium] hylemonae strain TN-271, Bacteroides mediterraneensis strain Marseille-P2644, Enterorhabdus muris strain WCA-131-CoC-2, Eubacterium pyruvativorans strain 1-6, Monoglobus pectinilyticus strain 14, Cloacibacillus porcorum strain CL-84, Harryflintia acetispora strain V20-281a, Collinsella massiliensis strain GD3, Denitrobacterium detoxificans strain NPOH1, or Novibacillus thermophiles strain SG-1.

12. The method of claim 9, wherein the measured biomarker is a biomarker associated with a bacterium selected from the group consisting of Eubacterium, Monoglobus and Cloacibacillus, wherein the female cow is bred if the biomarker associated with Eubacterium is not detected, and wherein the female cow is bred if the biomarker associated with the presence of at least one of Monoglobus and Cloacibacillus is detected.

13. The method of claim 9, wherein the step of measuring the level of a biomarker comprises:
c) detecting a protein associated with a particular bacterium; or
d) detecting a nucleic acid associated with a particular bacterium.

14. The method of claim 13, wherein the nucleic acid is a component of a 16S or 23S ribosomal subunit.

15. The method of claim 14, wherein the nucleic acid comprises a sequence selected from the group consisting of SEQ ID NOs: 1-30.

16. The method claim 9, wherein the sample is collected from a female cow prior to estrus synchronization, during estrus synchronization, prior to the onset of estrus, or prior to breeding.

17. A kit comprising reagents used to detect the presence or relative abundance of at least 2 biomarkers associated with bacteria of the following species in vaginal swab samples collected from a female cow: Histophilus somni, Colidextribacter massiliensis, Campylobacter lanienae, Bacteroides plebeius, Bacteroides xylanolyticus, Ihubacter massiliensis, Intestinimonas butyriciproducens, Merdimonas faecis, Ruminococcus lactaris, Lactonifactor longoviformis, Oscillibacter ruminantium, and [Clostridium] cellobioparum.

18. The kit of claim 17, wherein at least one of the measured biomarkers is associated with a bacterium of the following strains: Histophilus somni strain 8025, Colidextribacter massiliensis strain Marseille-P3083, Campylobacter lanienae strain CCUG, Oscillibacter ruminantium strain GH1, Bacteroides plebeius strain M12, Ihubacter massiliensis strain Marseille, Intestinimonas butyriciproducens strain SRB-521-5-I, Bacteroides xylanolyticus strain X5-1, Merdimonas faecis strain BR31, Ruminococcus lactaris strain ATCC, [Clostridium] cellobioparum strain DSM 1351, or Lactonifactor longoviformis strain ED-Mt61/PYG-s6.

19. The kit of claim 17, wherein the presence or absence of the bacterial species Campylobacter lanienae, Merdimonas faecis, or Lactonifactor longoviformis is assessed qualitatively.

20. The kit of claim 17, wherein the kit further comprises:
a) antibodies specific to proteins associated with particular bacteria; or
b) sets of PCR primers that amplify nucleic acids associated with particular bacteria.

21. The kit of claim 20, wherein the nucleic acids comprise at least one sequence selected from the group consisting of SEQ ID NOs: 1-30.

22. A kit comprising reagents used to detect the presence or relative abundance of at least 2 biomarkers associated with bacteria of the following species in fecal samples collected from a female cow: Bacteroides mediterraneensis, Enterorhabdus muris, Eubacterium pyruvativorans, Monoglobus pectinilyticus), Harryflintia acetispora, Collinsella massiliensis, Denitrobacterium detoxificans, Parapedobacter lycopersici, Parapedobacter soli, [Clostridium] hylemonae, Cloacibacillus porcorum, and Novibacillus thermophiles.

23. The kit of claim 22, wherein at least one of the measured biomarkers is associated with a bacterium of the following strains: Parapedobacter lycopersici strain T16R-256, Parapedobacter soli strain DCY14, [Clostridium] hylemonae strain TN-271, Bacteroides mediterraneensis strain Marseille-P2644, Enterorhabdus muris strain WCA-131-CoC-2, Eubacterium pyruvativorans strain 1-6, Monoglobus pectinilyticus strain 14, Cloacibacillus porcorum strain CL-84, Harryflintia acetispora strain V20-281a, Collinsella massiliensis strain GD3, Denitrobacterium detoxificans strain NPOH1, or Novibacillus thermophilus strain SG-1.

24. The kit of claim 22, wherein the presence or absence of the bacterial species Eubacterium pyruvativorans, Monoglobus or Cloacibacillus porcorum is assessed qualitatively.

25. The kit of claim 22, wherein the kit further comprises:
c) antibodies specific to proteins associated with particular bacteria; or
d) sets of PCR primers that amplify nucleic acids associated with particular bacteria.

26. The kit of claim 25, wherein the nucleic acids comprise at least one sequence selected from the group consisting of SEQ ID NOs: 1-30.
